(19) United States
(12) Patent Application Publication
(10) Pub. No.: US 2013/0196928 A1
(43) Pub. Date: Aug. 1, 2013

(54) LIBRARY FROM TOXIN MUTANTS AND METHODS OF USING SAME

(71) Applicant: Molecular Templates, Inc., Georgetown, TX (US)

(72) Inventors: Jean Gariepy, Toronto (CA); Leigh Revers, Toronto (CA)

(73) Assignee: MOLECULAR TEMPLATES, INC., Georgetown, TX (US)

(21) Appl. No.: 13/739,242

(22) Filed: Jan. 11, 2013

Related U.S. Application Data
(63) Continuation of application No. 12/088,206, filed on Nov. 19, 2008, now abandoned.
(60) Provisional application No. 60/720,924, filed on Sep. 26, 2005.

(51) Int. Cl. C07K 14/245

(52) U.S. Cl. CPC C07K 14/245
USPC 514/19.3; 530/350; 530/410

(57) ABSTRACT

This application relates to ABx toxin mutants and libraries of said mutant proteins, in which a peptide insert is introduced into the protease-sensitive loop of the A-chain sequence to alter the type of cells to which toxic species are delivered. Said libraries are used in the development of therapeutics targeted against specific cell types.

### CROSS-REFERENCE TO RELATED APPLICATIONS

This application is a continuation of U.S. application Ser. No. 12/088,206, which is a National Stage of International Application PCT/CA2006/001581, filed Sep. 26, 2006, which claims the benefit of U.S. Provisional Application Ser. No. 60/720,924, filed Sep. 26, 2005, each of which is incorporated by reference herein in its entirety.

### SEQUENCE LISTING

The instant application contains a Sequence Listing which has been submitted in ASCII format via EFS-Web and is hereby incorporated by reference in its entirety. Said ASCII Copy, created on Mar. 28, 2012, is named 10741500.txt and is 191,069 bytes in size.

### BACKGROUND OF THE INVENTION

This application relates to libraries of toxin mutants, and to methods of using same in the development of therapeutics targeted against specific cell types.

Plant and bacterial toxins have a structural organization with two or more polypeptide domains or subunits responsible for distinct functions, referred to as A and B. The toxins may be referred to as ABx toxins where x represents the number of identical or homologous B subunits in the toxin. This family of framework-related toxins includes examples such as Shiga and Shiga-like toxins, the E. coli heat-labile enterotoxins, cholera toxin, diphtheria toxin, pertussis toxin, Pseudomonas aeruginosa exotoxin A (Olsnes, S, and Sandvik, K. (1988) in Immunotoxins pp. 39-73, Kluwer Academic, Boston; Sandvik, K., Dubinina, E., Garred, O., et al. (1992) Biochem. Soc. Trans. 20:724) as well as plant toxins such as ricin and abrin. In some cases the toxin are heteromeric, in that the B chains are actually separate entities that connect to the toxic A chain via a non-covalent bonding. In other cases, the toxin is monomelic, since the B chain is part of the same protein when the toxin is produced in nature. In many cases, the A chain has been characterized as having two domains, an A1 domain and an A2 domain.

Based on their ability to block protein synthesis, proteins such as Shiga and Shiga-like toxins as well as ricin, abrin, gelonin, crotin, pokeweed antiviral protein, saporin, momordin, modeccin, sarcin, diphtheria toxin and exotoxin A have been referred to as ribosome-inactivating proteins (RIP). The potency of RIPs is exceedingly high; one molecule of diphtheria toxin A chain (Yamaizumi, et al. (1978) Cell 15:245-250) or ricin A chain (Eiklid, et al. (1980) Exp. Cell Res. 126:321-326) having been shown to be sufficient to kill a eukaryotic cell.

International Patent Publication No. WO99/40185 describes libraries of mutant toxins in which mutations are introduced into the binding domain to alter the type of cells to which the toxic species are delivered. The new proteins are derived by mutating a binding subunit of the wild type heteromeric protein cytotoxic protein to create a library of microorganism clones producing mutant proteins, which are then screened for the ability to specifically bind to and kill a target cell type.

U.S. Pat. No. 5,552,144 discloses a Shigella-like toxin II variant to which a mutation is introduced into the A chain at position 167 to change the amino acid at this position to one with a different charge. This resulted in a toxin with less of the enzymatic activity associated with toxicity.

U.S. Pat. No. 6,593,132 describes recombinant toxic proteins which are specifically toxic to diseased cells but do not depend for their specificity of action on a specific cell binding component. The recombinant proteins of the '132 patent have an A chain of a ricin-like toxin linked to a B chain by a synthetic linker sequence which may be cleaved specifically by a protease localized in cells or tissues affected by a specific disease to liberate the toxic A chain thereby selectively inhibiting or destroying the diseased cells or tissues.

U.S. Pat. No. 6,649,742 discloses Type I ribosome-inactivating proteins (RIPs) and analogs of the RIPs having a cysteine available for disulfide bonding to targeting molecules. The RIPs and RIP analogs are used as components of cytotoxic therapeutic agents to selectively eliminate any cell type to which the RIP component is targeted by the specific binding capacity of the second component of the agent.

PCT application PCT/CA2004/000433 by the present inventors discloses combinatorial protein libraries comprising a plurality of protein species, in which each protein species comprises an A chain of a heteromeric toxic protein, into which an insert has been introduced.

### SUMMARY OF THE INVENTION

An aspect of the present invention is a combinatorial protein library comprising a plurality of protein species, each protein species comprising an A chain of a toxic protein into which an insert has been introduced, wherein, (a) the insert is a polypeptide of varying amino acid sequence having a length of at least 2 amino acid residues; and (b) the insert is introduced into the protease-sensitive loop of the A chain sequence. In a further embodiment, the library may comprise up to or over 100 protein species, and may be formed by introducing the insert into a Shiga-like toxin IA chain, for example, between amino acids 242 and 261, such as between amino acids 245 and 246.

In a further embodiment of the present invention, the insert has a length of 7 amino acids.

Another aspect of the present invention is a mutant protein comprising an A chain of a toxic protein into which an insert has been introduced, wherein the insert is a polypeptide of varying amino acid sequence having a length of at least 2 amino acid residues; and the insert is introduced into the protease-sensitive loop of the A chain sequence. In a further embodiment, the A chain can be a Shiga-like toxin I A chain. In a further embodiment, the insert may be introduced between amino acids 242 and 261, such as between amino acids 245 and 246.

A further aspect of the present invention is a method for identifying a ligand that bind to a specific target/receptor, comprising the steps of exposing cells known to possess the target/receptor to members of a combinatorial protein library as described herein; selecting members of the protein library which are observed to be toxic to the cells; evaluating the selected members of the protein library to determine the sequence of the inserted region, whereby a peptide of the sequence of the inserted region is identified as a possible ligand for a target/receptor on the cell; and further testing peptides of the sequence of the inserted region to confirm that they are a ligand for the specific target/receptor.

A further aspect of the present invention is a method for isolating a toxin specific for a known target/receptor comprising the steps of: exposing the target/receptor to a combinatorial protein library as described herein; and isolating at least one protein species from the combinatorial protein library captured by binding to the target/receptor.

A further aspect of the present invention is a combinatorial protein library comprising a plurality of protein species, each protein species comprising a modified A chain of a toxic protein into which an insert has been introduced, wherein, the modified A chain comprises a wild-type A chain containing a mutation in at least one cysteine; the insert is a polypeptide of varying amino acid sequence having a length of at least 2 amino acid residues; and the insert is introduced into the region which would be a protease-sensitive loop in the wild-type A chain sequence.

A further aspect of the present invention is a mutant protein comprising a modified A chain of a toxic protein into which an insert has been introduced, wherein, the modified A chain comprises a wild-type A chain containing a mutation in at least one cysteine; the insert is a polypeptide of varying amino acid sequence having a length of at least 2 amino acid residues; and the insert is introduced into a region which would be a protease-sensitive loop in the wild type A chain sequence. In a further embodiment, the modified A chain further comprises a label bound to a cysteine.

Another embodiment of the present invention is a polypeptide sequence selected from the group consisting of the polypeptide sequence listed in FIG. 11C, 11D, 12A, 12B, 12C, or 12D.

Another embodiment of the present invention is a method for identifying a ligand that bind to a specific target/receptor, comprising the steps of: exposing cells known to possess the target/receptor to members of a combinatorial protein library as described herein; selecting members of the protein library which are observed to be toxic to the cells; evaluating the selected members of the protein library to determine the sequence of the inserted region, whereby a peptide of the sequence of the inserted region is identified as a possible ligand for a target/receptor on the cell; and further testing peptides of the sequence of the inserted region to confirm that they are a ligand for the specific target/receptor.

Another embodiment of the present invention is a method for isolating a toxin specific for a known target/receptor comprising the steps of: exposing the target/receptor to a combinatorial protein library as described herein; and isolating at least one protein species from the combinatorial protein library captured by binding to the target/receptor.

Another embodiment of the present invention is a combinatorial protein library comprising a plurality of protein species, each protein species comprising an A1 chain of a toxic protein into which an insert has been introduced, wherein, the insert is a polypeptide of varying amino acid sequence having a length of at least 2 amino acid residues; and the insert is introduced into the portion of the A1 chain that would be a protease-sensitive loop if the A1 chain was a full-length A chain.

Another embodiment of the present invention is a mutant protein comprising an A1 chain of a toxic protein into which an insert has been introduced, wherein, the insert is a polypeptide of varying amino acid sequence having a length of at least 2 amino acid residues; and the insert is introduced into the portion of the A1 chain that would be a protease-sensitive loop if the A1 chain was a full length A chain.

Another embodiment of the present invention is a method for identifying a ligand that bind to a specific target/receptor, comprising the steps of: exposing cells known to possess the target/receptor to members of a combinatorial protein library as described herein; selecting members of the protein library which are observed to be toxic to the cells; evaluating the selected members of the protein library to determine the sequence of the inserted region, whereby a peptide of the sequence of the inserted region is identified as a possible ligand for a target/receptor on the cell; and further testing peptides of the sequence of the inserted region to confirm that they are a ligand for the specific target/receptor. Another embodiment of the present invention is a method for isolating a toxin specific for a known target/receptor comprising the steps of: exposing the target/receptor to a combinatorial protein library as disclosed herein; and isolating at least one protein species from the combinatorial protein library captured by binding to the target/receptor.

A further embodiment of the present invention is a combinatorial protein library comprising a plurality of protein species, each protein species comprising a modified A1 chain of a toxic protein into which an insert has been introduced, wherein, the modified A1 chain comprises a wild-type A1 chain containing a mutation in one cysteine; the insert is a polypeptide of varying amino acid sequence having a length of at least 2 amino acid residues; and the insert is introduced into the portion of the A1 chain that would be a protease-sensitive loop if the A1 chain was a full length A chain.

A further embodiment of the present invention is a mutant protein comprising a modified A1 chain of a toxic protein into which an insert has been introduced, wherein, the modified A1 chain comprises a wild-type A1 chain containing a mutation in one cysteine; the insert is a polypeptide of varying amino acid sequence having a length of at least 2 amino acid residues; and the insert is introduced into the portion of the A1 chain that would be a protease-sensitive loop if the A1 chain was a full-length A chain.

A further embodiment of the present invention is a method for identifying a ligand that bind to a specific target/receptor comprising the steps of: exposing cells known to possess the target/receptor to members of a combinatorial protein library as disclosed herein; selecting members of the protein library which are observed to be toxic to the cells; evaluating the selected members of the protein library to determine the sequence of the inserted region, whereby a peptide of the sequence of the inserted region is identified as a possible ligand for a target/receptor on the cell; and further testing peptides of the sequence of the inserted region to confirm that they are a ligand for the specific target/receptor. A further embodiment of the present invention is a method for isolating a toxin specific for a known target/receptor comprising the steps of: exposing the target/receptor to a combinatorial protein library as disclosed herein; and isolating at least one protein species from the combinatorial protein library captured by binding to the target/receptor.

In another aspect of the present invention, the combinatorial protein library can have the insert introduced at the N-terminus of the A chain sequence instead of in the protease sensitive loop region.

Another embodiment of the present invention is a method for identifying a ligand that bind to a specific target/receptor, comprising the steps of: exposing cells known to possess the target/receptor to members of a combinatorial protein library as disclosed herein; selecting members of the protein library which are observed to be toxic to the cells; evaluating the selected members of the protein library to determine the sequence of the inserted region, whereby a peptide of the sequence of the inserted region is identified as a possible ligand for a target/receptor on the cell; and further testing peptides of the sequence of the inserted region to confirm that they are a ligand for the specific target/receptor.

Another embodiment of the present invention is a method for isolating a toxin specific for a known target/receptor comprising the steps of: exposing the target/receptor to a combinatorial protein library as disclosed herein; and isolating at least one protein species from the combinatorial protein library captured by binding to the target/receptor.

### BRIEF DESCRIPTION OF THE DRAWINGS

FIG. 1 is a schematic diagram depicting the A1 and A2 domains of wild-type SLT-I. The A chain is composed of 293 amino acids. The chain is cleaved by furin to produce an A1 catalytic fragment and an A2 C-terminal tail non-covalently associated with the B pentamer. A protease-sensitive loop (stripped area) is defined by the only two cysteine residues in the A chain (Cys 242 and 261). Tyr77, Glu167, Arg170 and Trp203 represent residues important for the catalytic activity of the A1 domain (arrows).

FIG. 2A is a schematic representation of the SLT-I A chain (1-293) with the breast cancer-associated MUCl epitope PDTRPAP (SEQ ID NO:87) (control sequence recognized by the mAb One M27) inserted between residues 245 and 246 and a 6-Histidine tag (SEQ ID NO: 89) followed by a protease cleavage site (such as a furin or thrombin cleavage site) at its N-terminus.

FIG. 2 B is a depiction of a SLT-I A chain-tripeptide library construction where the three positions of the MUCl epitope recognized by the mAb One M27 are randomized (XXX region). The tripeptide library was inserted in a naturally occurring loop region of the A chain created by the presence of a disulfide bridge between Cys 242 and Cys 261. Figure discloses “PDXXXAP” SEQ ID NO: 88.

FIG. 2 C is a depiction of the SLT-I A chain tripeptide library construction of FIG. 2 B, wherein cysteine 261 has been mutated to an alanine. Note that in this case (as in many of the figures below) the disulfide bridge present in FIG. 28 can not be formed due to the absence of one of the cysteines. Figure discloses “PDXXXAP” as SEQ ID NO: 88.

FIG. 2D is a depiction of the SLT-I A chain tripeptide library construction of FIG. 2B, wherein cysteine 242 has been mutated to an alanine. Figure discloses “PDXXXAP” as SEQ ID NO: 88.

FIG. 2E is a depiction of the SLT-I A chain tripeptide library construction of FIG. 28, wherein cysteine 242 and cysteine 261 have both been mutated to alanine residues. Figure discloses “PDXXXAP” as SEQ ID NO: 88.

FIG. 2F is a depiction of the SLT-I A chain tripeptide library construction of FIG. 2C, wherein the C242 residue has been biotinylated. Figure discloses “PDXXXAP” as SEQ ID NO: 88.

FIG. 2G is a schematic representation of the SLT-I A1 chain (1-251) with the breast cancer-associated WIC 1 epitope PDIRPAP (SEQ ID NO: 87) (control sequence recognized by the mAb One M27) inserted between residues 245 and 246 and a Histidine tag followed by a protease cleavage site (such as a furin or thrombin cleavage site) at its N-terminus. Figure discloses “PDXXAP” as SEQ ID NO: 88.

FIG. 2 H is a depiction of our SLT-I chain-tripeptide library construction where the three key positions of the MUCl epitope recognized by the mAb One M27 were randomized (XXX region). Figure discloses “PDXXAP” as SEQ ID NO: 88.

FIG. 2 I is a depiction of the SLT-I A1 chain tripeptide library construction of FIG. 2 H, wherein cysteine 242 has been mutated to an alanine. Figure discloses “PDXXAP” as SEQ ID NO: 88.

FIG. 2 I is a depiction of the SLT-I A1 chain tripeptide library construction of FIG. 2H, wherein cysteine 242 has been biotinylated. Figure discloses “PDXXAP” as SEQ ID NO: 88.

FIG. 3 shows a representative ELISA data set from screening 96 distinct single A chain variants from our SLT-I A chain-tripeptide library with tnAb One M27. Toxin variant #41 (Tables 1 and 2) gave a strong ELISA signal and had the expected epitope.

FIG. 4A shows a schematic diagram of a 7-amino acid random segment inserted between residues 245 and 246 of the A chain.

FIG. 4B shows a schematic diagram of a 7-amino acid random segment inserted between residues 245 and 246 of the A chain, wherein the cysteine at position 261 has been mutated to an alanine.

FIG. 4C shows a schematic diagram of a 7-amino acid random segment inserted between residues 245 and 246 of the A chain, wherein the cysteine at position 242 has been mutated to an alanine. FIG. 4D shows a schematic diagram of a 7-amino acid random segment inserted between residues 245 and 246 of the A chain, wherein the cysteine at positions 242 and 261 have been mutated to alanines.

FIG. 4E shows a schematic diagram of a 7-amino acid random segment inserted between residues 245 and 246 of the A chain, wherein the cysteine at position 261 has been mutated to an alanine and the cysteine at position 242 has been biotinylated.

FIG. 4F shows a schematic diagram of a 7-amino acid random segment inserted between residues 245 and 246 of the A1 chain.

FIG. 4G shows a schematic diagram of a 7-amino acid random segment inserted between residues 245 and 246 of the A1 chain, wherein the cysteine residue at position 242 has been biotinylated.

FIG. 4H shows a schematic diagram of a 7-amino acid random segment inserted between residues 245 and 246 of the A1 chain, wherein the cysteine residue at position 242 has been mutated to an analine.

FIG. 5 shows the results of tests on seven toxin variants that were identified as repeatable killers of the human melanoma cell line 518A2. The abscissa represents the log concentration of toxin used to treat the cells and the ordinate depicts the observed percentage of cells that are viable after 48 hours. The closed triangles depicts the effect of the wild type toxin on 518A2 cells while the two most efficacious A chain variants were termed SAM#3 (open squares) and SAM#5 (X symbols).

FIG. 6 shows the amino acid seq uence (SEQ ID NO: 6) corresponding to the wild-type SLT-I protein of FIG. 1. Amino acid sequences are shown from N-terminus to C-terminus.

FIG. 7A shows the amino acid sequence (SEQ ID NO: 8) corresponding to the SLT-A chain of FIG. 2 A, less the N-terminus tags (the His-tag and the protease cleavage site).

FIG. 7B shows the amino acid sequence (SEQ ID NO: 9) corresponding to the SLT-A chain of FIG. 2A, with an example of one possible N-terminus tag, comprising a 8-His-tag and a protease cleavage site.

FIG. 7C shows the amino acid sequence (SEQ ID NO: 11) corresponding to the SLT-A chain-tripeptide library construction of FIG. 2B, less the N-terminus tags (the His-tag and the protease cleavage site).

FIG. 7D shows the amino acid sequence (SEQ ID NO: 12) corresponding to the SLT-A chain-tripeptide library construction of FIG. 2B, with an example of one possible N-terminus tag, comprising a 8-His-tag (SEQ ID NO: 90) and a protease cleavage site.

FIG. 8A shows the amino acid sequence (SEQ ID NO: 14) corresponding to the SLT-I A chain tripeptide library of FIGS. 2C and 2F, less the N-terminus tags (the His-tag and the protease cleavage site).

FIG. 8B shows the amino acid sequence (SEQ ID NO: 93) corresponding to the SLT-I A chain tripeptide library of FIGS. 2C and 2F, with an example of one possible N-terminus tag, comprising a 8-His-tag (SEQ ID NO: 90) and a protease cleavage site.

FIG. 8C shows the amino acid sequence (SEQ ID NO: 17) corresponding to the SLT-I A chain tripeptide library of FIG. 2D, less the N-terminus tags (the His-tag and the protease cleavage site).

FIG. 8D shows the amino acid sequence (SEQ ID NO: 18) corresponding to the SLT-I A chain tripeptide library of FIG. 2D, with an example of one possible N-terminus tag, comprising a 8-His-tag and a protease cleavage site.

FIG. 9A shows the amino acid sequence (SEQ ID NO: 20) corresponding to the SLT-I A chain tripeptide library of FIG. 2E, less the N-terminus tags (the His-tag and the protease cleavage site).

FIG. 9B shows the amino acid sequence (SEQ ID NO: 21) corresponding to the SLT-I A chain tripeptide library of FIG. 2E, with an example of one possible N-terminus tag, comprising a 8-His-tag (SEQ ID NO: 90) and a protease cleavage site.

FIG. 9C shows the amino acid sequence (SEQ ID NO: 23) corresponding to the SLT-I A1 chain of FIG. 2G, less the N-terminus tags (the His-tag and the protease cleavage site).

FIG. 9D shows the amino acid sequence (SEQ ID NO: 24) corresponding to the SLT-A1 chain of FIG. 2G, with an example of one possible N-terminus tag, comprising a 8-His-tag (SEQ ID NO: 90) and a protease cleavage site.

FIG. 10A shows the amino acid sequence (SEQ ID NO: 94) corresponding to the SLT-I A1 chain of FIGS. 2H and 2J, less the N-terminus tags (the His-tag and the protease cleavage site).

FIG. 10B shows the amino acid sequence (SEQ ID NO: 95) corresponding to the SLT-I A1 chain of FIGS. 2H and 2J, with an example of one possible N-terminus tag, comprising a 8-His-tag (SEQ ID NO: 90) and a protease cleavage site.

FIG. 10C shows the amino acid sequence (SEQ ID NO: 96) corresponding to the SLT-I A1 chain of FIG. 21, less the N-terminus tags (the His-tag and the protease cleavage site).

FIG. 10D shows the amino acid sequence (SEQ ID NO: 30) corresponding to the SLT-I A1 chain of FIG. 2I, with an example of one possible N-terminus tag, comprising a 8-His-tag (SEQ ID NO: 90) and a protease cleavage site.

FIG. 11A shows the amino acid sequence (SEQ ID NO: 32) corresponding to the SLT-I A chain of FIG. 4 A, less the N-terminus tags (the His-tag and the protease cleavage site).

FIG. 11B shows the amino acid sequence (SEQ ID NO: 33) corresponding to the SLT-I A chain of FIG. 4A, with an example of one possible N-terminus tag, comprising a 8-His-tag (SEQ ID NO: 90) and a protease cleavage site.

FIG. 11C shows the amino acid sequence (SEQ ID NO: 35) corresponding to the SLT-I A chain of FIGS. 4B and 4E, less the N-terminus tags (the His-tag and the protease cleavage site).

FIG. 11D shows the amino acid sequence (SEQ ID NO: 36) corresponding to the SLT-I A chain of FIGS. 4B and 4E, with an example of one possible N-terminus tag, comprising a 8-His-tag (SEQ ID NO: 90) and a protease cleavage site.

FIG. 12A shows the amino acid sequence (SEQ ID NO: 38) corresponding to the SLT-I A chain of FIG. 4C, less the N-terminus tags (the His-tag and the protease cleavage site).

FIG. 12B shows the amino acid sequence (SEQ ID NO: 39) corresponding to the SLT-I A chain of FIG. 4C, with an example of one possible N-terminus tag, comprising a 8-His-tag (SEQ ID NO: 90) and a protease cleavage site.

FIG. 12C shows the amino acid sequence (SEQ ID NO: 41) corresponding to the SLT-I A chain of FIG. 4D, less the N-terminus tags (the His-tag and the protease cleavage site).

FIG. 12D shows the amino acid sequence (SEQ ID NO: 42) corresponding to the SLT-I A chain of FIG. 4D, with an example of one possible N-terminus tag, comprising a 8-His-tag (SEQ ID NO: 90) and a protease cleavage site.

FIG. 13A shows the amino acid sequence (SEQ ID NO: 44) corresponding to the SLT-I A1 chain of FIGS. 4F and 4G, less the N-terminus tags (the His-tag and the protease cleavage site).

FIG. 13B shows the amino acid sequence (SEQ ID NO: 45) corresponding to the SLT-I A1 chain of FIGS. 4F and 4G, with an example of one possible N-terminus tag, comprising a 8-His-tag (SEQ ID NO: 90) and a protease cleavage site.

FIG. 13C shows the amino acid sequence (SEQ ID NO: 47) corresponding to the SLT-I A1 chain of FIG. 4H, less the N-terminus tags (the His-tag and the protease cleavage site).

FIG. 13D shows the amino acid sequence (SEQ ID NO: 48) corresponding to the SLT-I A1 chain of FIG. 4H, with an example of one possible N-terminus tag, comprising a 8-His-tag (SEQ ID NO: 90) and a protease cleavage site.

FIG. 14A shows the amino acid sequence (SEQ ID NO: 50) corresponding to the SLT-I A region, with cysteine 261 mutated to an alanine.

FIG. 14B shows the amino acid sequence (SEQ ID NO: 51) corresponding to the SLT-I A region, with cysteine 261 mutated to an alanine, and with an example of one possible N-terminus tag, comprising an 8-HIS-tag (SEQ ID NO: 90) and a protease cleavage site.

FIG. 14C shows the amino acid sequence (SEQ ID NO: 53) corresponding to the SLT-I A region, with cysteine 242 mutated to an alanine.

FIG. 14D shows the amino acid sequence (SEQ ID NO: 34) corresponding to the SLT-I A region, with cysteine 242 mutated to an alanine, and with an example of one possible N-terminus tag, comprising an 8-HIS-tag (SEQ ID NO: 90) and a protease cleavage site.

FIG. 15A shows the amino acid sequence (SEQ ID NO: 56) corresponding to the SLT-I A region, with cysteines 242 and 261 mutated to alanines.

FIG. 15B shows the amino acid sequence (SEQ ID NO: 57) corresponding to the SLT-I A region, with cysteines 242 and 261 mutated to alanines, and with an example of one possible N-terminus tag, comprising an 8-HIS-tag (SEQ ID NO: 90) and a protease cleavage site.

FIG. 15C shows the amino acid sequence (SEQ ID NO: 59) corresponding to the SLT-I A1 region.

FIG. 15D shows the amino acid sequence (SEQ ID NO: 60) corresponding to the SLT-I A1 region with an example of one possible N-terminus tag, comprising an 8-HIS-tag (SEQ ID NO: 90) and a protease cleavage site.

FIG. 16A shows the amino acid sequence (SEQ ID NO: 62) corresponding to the SLT-I A1 region, with cysteine 242 mutated to an alanine.

FIG. 16B shows the amino acid sequence (SEQ ID NO: 63) corresponding to the SLT-I A1 region with cysteine 242 mutated to an alanine, and with an example of one possible N-terminus tag, comprising an 8-HIS-tag (SEQ ID NO: 90) and a protease cleavage site.

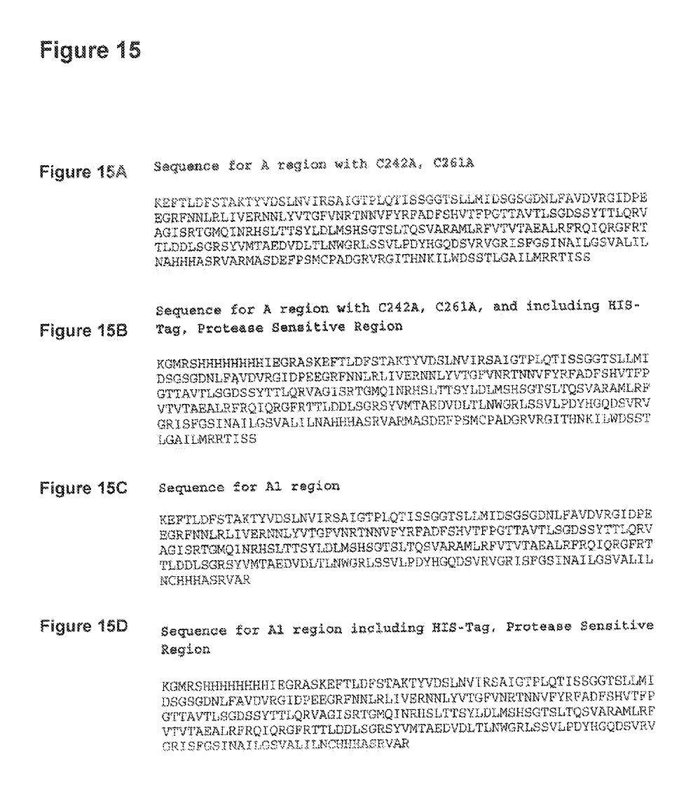

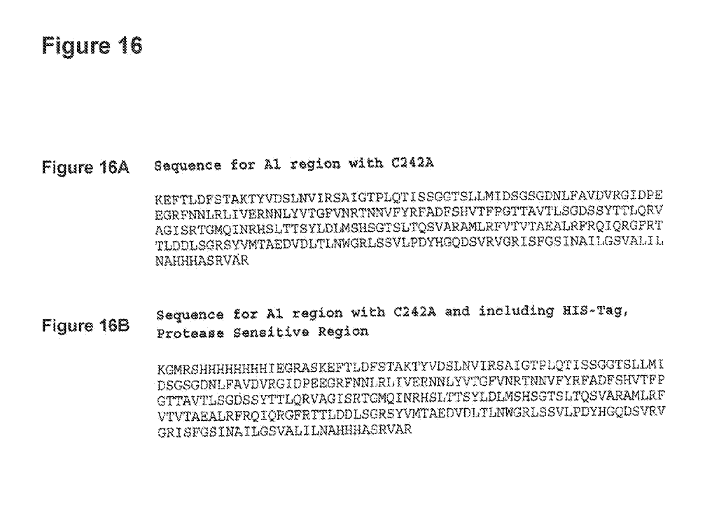

For FIGS. 6-16 that include the N-terminus tag, the tag can be varied as needed; for example, the tag KGMRSHHHHHHHHRVARAS (SEQ ID NO: 91) can be used instead of KGMRSHHHHHHHHIEGRAS (SEQ ID NO: 92).

### DETAILED DESCRIPTION OF THE INVENTION

The present invention relates to a combinatorial protein library comprising a plurality of protein species, each protein species comprising an A or A1 chain of a heteromeric toxic protein into which an insert has been introduced. The insert is a polypeptide of varying amino acid sequence having a length of 2 or more amino acid residues, for example from 3 to 200 amino acid residues; and is introduced into the protease-sensitive loop of the A chain sequence, or the equivalent remaining area in the A1 chain sequence. The library provides a collection of protein species that can be screened for individual proteins that are toxic against specific cell types, such as specific cancer cell types. Individual protein species thus selected are suitably used in the treatment of the cancer.

As used in the specification and claims of this application, the term “combinatorial library” refers to a mixture of species each of which has a common portion and a variable portion. In the case of a “combinatorial protein library” each of the species is a protein or peptide, and the common portions and the variable portions are each amino acid sequences. In the case of a combinatorial expression library, the species are microorganisms, expression vectors or polynucleotides which, when expressed, produce proteins or peptides having common portions and variable portions. In this case, the common portions and the variable portions are each nucleotide sequences. Since the purpose of the combinatorial library is to provide multiple variants for screening purposes, the combinatorial library preferably contains at least 100 distinct species of protein or expression unit, more preferably at least 1000 distinct species.

As used in the specification and claims of this application, the term “heteromeric toxic protein” refers to the class of protein toxins with the common organization theme of being heteromeric in nature with two or more polypeptide domains or subunits responsible for distinct functions (Merritt, E. A., and Hol, W. G. J. (1995) Curr. Opin. Struct. Biol. 5:165 1). In such proteins, the two or more subunits or domains could be referred to as A and B, and the toxins as ABx toxins where x represents the number of identical or homologous B subunits in the toxin. This family of framework-related toxins includes examples such as Shiga and Shiga-like toxins, the E. coli heat-labile enterotoxins, cholera toxin, diphtheria toxin, pertussis toxin, Pseudomonas aeruginosa exotoxin A as well as plant toxins such as ricin and abrin. Based on their ability to block protein synthesis, proteins such as Shiga and Shiga-like toxins as well as ricin, abrin, gelonin, crotin, pokeweed antiviral protein, saporin, momordin, modeccin, sarcin, diphtheria toxin and exotoxin A have been referred to as ribosome-inactivating proteins (RIP), hi these naturally-occurring heteromeric toxic proteins, the A chain is the toxic portion, while the B chains form a binding moiety which binds to a receptor on a cell susceptible to the toxin, thereby delivering the A chain to the cell, hi many cases, the A chain has been further characterized as having two domains, an A1 domain and an A2 domain. Typically, the A1 domain includes the cytotoxic elements of the A subunit and a portion of the protease-sensitive loop portion, but often does not include the B chain binding portion.

One specific example of the A chain of a heteromeric toxic protein is the A chain of SLT-I which has the sequence given is Seq. ID No.1. The A chain of SLT-I comprises of 293 amino acids with the enzymatic (toxic) domain spanning residues 1 to 239. A protease sensitive loop encompassing residues 242 to 261 is normally exposed, and is a suitable site for inserting a peptide sequence. The A chain of SLT-I is characterized as having an A1 domain, spanning residues 1 to 251, and including the entire enzymatic (toxic domain, and an A2 domain spanning residues 252-261.

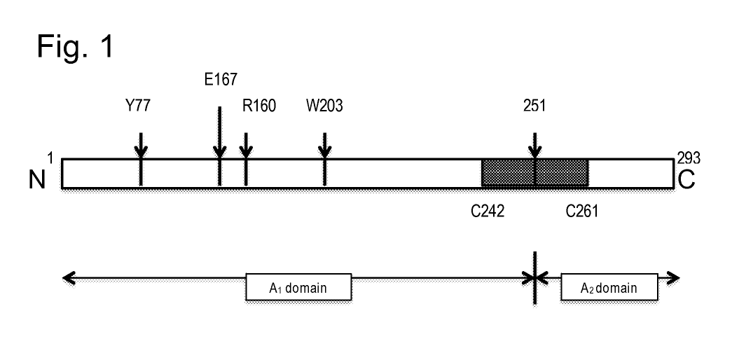

SLT-I is a type II ribosome inactivating protein produced by pathogenic strain of Escherichia coli (0157:H7) (24). SLT-I is an A13:5 complex of about 70 kl) (O'Brien, A. D., and Holmes, R. K. (1987) Shiga and Shiga-like toxins. Microbiol Rev 51, 206-220.). The single 32 kD catalytic A subunit is non-covalently associated with a pentamer of five identical 7.7 kD B subunits. The B subunit pentamer recognizes the glycolipid globotriaosylceramide (also known as CD77 or Gb3) on the surface of target cells (Lingwood, C. A. (1993) Verotoxins and their glycolipid receptors. Adv Lipid Res 25, 189-211; jacewicz, et al. (1986) Pathogenesis of shigella diarrhea. XI. Isolation of a shigella toxin-binding glycolipid from rabbit jejunum and HeLa cells and its identification as globotriaosylceramide. J Exp Med 163, 1391-1404). A protease-sensitive loop located between Cys242 and 261 at the C terminus of the A chain is cleaved by furin during cellular routing (FIG. 1). The A chain remains associated with its B subunit pentamer due to an intrachain disulfide bond between Cys242 and Cys261 as it travels to the ER lumen (Sandvig, et al. (1989) Endocytosis from coated pits of Shiga toxin: a glycolipid-binding protein from Shigella dysenteriae 1. J Cell Biol 108, 1331-1343; 32. Garred, et al. (1995) Role of processing and intracellular transport for optimal toxicity of Shiga toxin and toxin mutants. Exp Cell Res 218, 39-49.). The disulfide bond is finally reduced in the ER lumen and the A1 chain (first 251 aa) is released and subsequently retrotranslocated to the cytosol where it inactivates ribosomes (O'Brien, et al. (1992) Shiga toxin: biochemistry, genetics, mode of action, and role in pathogenesis. Curr Top Microbiol Immunol 180, 65-94.). More specifically, the A chain of SLT-I is a N-glycosidase that catalytically cleaves a specific adenine nucleotide (4324) from the 28 S rRNA (Brigotti, et al. (1997) The RNA-N-glycosidase activity of Shiga-like toxin 1: kinetic parameters of the native and activated toxin. Toxicon 35, 1431-1437.). This event leads to the inhibition of protein synthesis by preventing the binding of aminoacyl tRNAs to the ribosome and halting protein elongation. Mutagenesis studies as well as structural analysis performed on the A chains of ST and ricin have delineated key conserved residues involved in catalytic activity (Deresiewicz, et al. (1992) Mutations affecting the activity of the Shiga-like toxin I A-chain. Biochemistry 31, 3272-3280; Ready, et al. (1991) Site-directed mutagenesis of ricin A-chain and implications for the mechanism of action. Proteins 10, 270-278). Residues important for catalytic activity of SLT-I are tyrosine 77, glutamic acid 167, arginine 170 and tryptophan 203 (Hovde, et al. (1988) Evidence that glutamic acid 167 is an active-site residue of Shiga-like toxin I. Proc Natl Acad Sci USA 85, 2568-2572; Yamasaki, et al. (1991) Importance of arginine at position 170 of the A subunit of Vero toxin 1 produced by enterohemorrhagic Escherichia coli for toxin activity. Microb Pathog 11, 1-9). In addition, binding of the toxin to the cell surface is important to introduction into the cell and thus for toxic activity. Because of this the A chain alone is not significantly toxic. In addition to the A or A1 chain of SLT-I, other toxins may also be used to form libraries and compositions in accordance with the invention, and may be used in the methods of the invention. Specifically, Shiga and other Shiga-like toxins, as well as ricin, abrin, gelonin, crotin, pokeweed antiviral protein, saporin, momordin, modeccin, sarcin, diphtheria toxin and exotoxin A, and other functionally-related ribosome inactivating proteins (RIP) may be used.

For purposes of making the combinatorial library of the invention, short amino acid sequences of at least 2 amino acids, for example of 3 to 200 amino acids residues in length, are inserted into this protease sensitive loop. The number of amino acids in the insert defines the number of possible random variants that can be in the library. For example, when the number of amino acids in the insert is 3, the maximum number of variants is 20<3> or 8000 variants. Larger inserts provide corresponding larger numbers of possible variants. It has also been found that certain other regions of the A or A1 chain may be used to insert the short amino acid sequences. The present inventors have shown, for example, that the N terminus region can support insertions of long amino acid sequences without significant loss of activity.

As an alternative to using inserts with purely random sequences, inserts can be designed based on a known template. For example, as described below in the context of Muc-1, variations in a sequence known to provide receptor binding properties for a particular cell type can be used to identify an insert that provides for optimization of the toxic properties of the protein construct. This same optimization may be performed on an individual sequence isolated by screening of a larger combinatorial library. It will be appreciated, however, that the insert in the proof of principle tests of Example 1 use an insert which is the target/receptor, while in the actual case the insert would be based on the sequence of a known ligand to be optimized for maximum effectiveness and specificity.

This approach, in which a specific, known receptor type is targeted illustrates a further aspect of the invention, namely a method for identifying peptide ligands that bind to specific targets receptors, such as tumor markers known to exist on cancer cells. In this method, a combinatorial protein library in accordance with the invention is screened against cells known to possess the target receptor. The toxin serves as the reporter, such that proteins which are shown to be toxic to the cells are evaluated to determine the sequence of the inserted region. Peptides of this sequence can then be used, in combination with a toxin or other molecules, to direct compounds to cells possessing the target/receptor. Further testing peptides of the sequence of the inserted region may be appropriate to confirm that they are a ligand for the specific target/receptor, as opposed to some other receptor on the cell type. This can be done using binding assays with isolated receptor, where such are available.

These inserts used in alternative to inserts with purely random sequences can use, as a known template, a small protein molecule with a known or desirable folded structure, for increasing sensitivity or specificity of binding. For example, an immunoglobulin like domain, such as a FC molecule sequence, a fragment of an FC molecule sequence, or a mimic of an FC molecule sequence, can be inserted. Libraries can therefore be created with a variable chain domain region of an FC molecule, or a random 7-mer (or other length random sequence) inserted in the variable region of an FC molecule, in turn inserted in the protease-sensitive loop region of the A or A1 molecule.

The invention also provides a method for identifying toxic substances specific for a known cell marker, and particularly markers that are available in isolated form. In this embodiment of the invention, the toxin need not serve as reporter. Thus, cells having the marker, or an isolated target/receptor, where available, are exposed to the combinatorial protein library. In preferred embodiments, the cells or the isolated target/receptor are immobilized on a solid support, such as in plastic wells. Captured proteins from the library are then rescreened against cells to confirm their toxicity and specificity for cells expressing the target/receptor, and their suitability for use as a therapeutic. This method can be used to identify toxins with binding inserts specific for any tumor marker or cell receptor, including without limitation tumor markers such as mucins such as MUC-I and its glycoforms Her-2, Her2-Neu, tyrosine kinase markers, EGFR, GD2, and GD3. Thus, in accordance with this specific aspect of the invention, a method for isolating a toxin specific for a known target receptor is provided that comprises the steps of:

(a) exposing the target/receptor to a combinatorial protein library comprising a plurality of protein species, each protein species comprising an A or A1 chain of a toxic protein into which an insert has been introduced, wherein, the insert is a polypeptide of varying amino acid sequence having a length of at least 2 amino acid residues; and the insert is introduced into the protease-sensitive loop of the A chain sequence or the equivalent portion of the A1 chain sequence; and

(b) isolating at least one protein species from the combinatorial protein library captured by binding to the target/receptor. As used in the specification and claims hereof, the term “isolating” refers to any mechanism for obtaining a composition containing the protein separated from the milieu in which it is expressed in a form suitable for further analysis. This would include release from the target/receptor following capture (for example by exposure to a competitive binding agent) or isolation from a culture of a clone expressing the protein found to be captured. The method may further comprises the step of screening the isolated protein against cells expressing the target/receptor, to confirm their toxicity for cells expressing the target/receptor. Procedures suitable for this screening are described in Example 3. As noted above, in this method, the target/receptor may be a purified target/receptor and may be immobilized on a solid support. The target/receptor may also be on the surface of cells, which may be immobilized. Where the target/receptor is on the surface of cells, the toxin can serve as a reporter, and the death of the cells is indicative of receptor binding.

Our experience with constructing SLT-I libraries has pointed out a number of practical issues that are appropriately considered in selecting a protein template for building combinatorial libraries. An important factor is to choose a single chain protein, preferably a bacterial protein of less than 300 amino acids (if the libraries are to be expressed in prokaryotes). Use of a smaller toxin both increases its potential to penetrate into solid tumors and reduces its immunogenicity. Secondly, the protein template should spontaneously fold in solution into its active form such that there is minimal need for host's chaperones. One should avoid for example scaffolds that contain multiple cysteine residues normally involved in disulfide bridges. In addition, a single chain protein as opposed to a multi-subunit complex may be more easily exported from bacteria. Thirdly, the protein template should possess an enzymatic activity, which can rapidly be measured to confirm the proper folding of peptide variants containing single and multiple site-directed mutations. Fourthly, a simple screening approach should be incorporated into the design of combinatorial libraries. Such searches should be amenable to high-throughput screening methods. The catalytic A chain of SLT-I (residues 1 to 293) and the A1 chain of SLT-I (residues 1-251) meet these criteria because they are a single chain that lack any known receptor binding function, and that have well defined structures and catalytic sites.

We have also found that cysteine 241 and 261 of the A chain of SLT-I can be replaced by site specific mutagenesis (or otherwise modified) in order to remove the single disulfide bridge present between these two cysteines in the A chain. Such advantageous mutations remove the need to reduce the peptide chain during purification and provide a simpler strategy for purifying single chain toxins comprising the A1 domain of A chain mutants. We have found that either cysteine 242 or cysteine 261, or both, can be mutated in this manner. The cysteine(s) can be mutated to alanine, though other mutations may result in the same desired effect while providing a suitable A or A1 chain.

An other advantage of mutating the cysteines in this manner results from the mutation of either of C242 or C261 (preferably, C261). This results in a shiga-like protein A chain with only one cysteine. The thiol group associated with this single cysteine residue is therefore free and offers the opportunity to introduce spectroscopic (derivatives of fluorescent chromophores that react with the thiol moiety), radioactive (thiol-reactive heavy ion complexes or metal chelators), or biochemical (for example, biotin) probe. These A chain variants, labeled with reporter probes, can then be used as agents for both in vitro and in vivo applications. For example, an inactivated single chain A or A1 variant, labeled with a reporter probe (and inactivated by mutating E 167 A and R1 70A) can be used as a non-toxic probe, as described above, to determine which patient population may benefit from being treated with which of the corresponding catalytically active single chain variants. A library of inactivated single chain A1 variants labeled in this manner can also be used to determine which binding domains (from said library) are effective, to determine which binding domains can be used in corresponding catalytically active single chain variants to treat a patient. Alternatively, these non-toxic probes can be used to determine which binding domains (from the randomly mutated library) can be used to target the desired cell population and used in either the toxic subunit, or bound to a separate and known therapeutic agent, to target and treat that cell population. Alternatively, once a specific desired library member has been identified, either through binding studies as described above or otherwise, that one specific library member (for example, an A1 chain with a specific desirable 7mer sequence inserted after amino acid 245) can be biotinylated for further purification or other studies.

Alternatively, the entire library can be biotinylated, allowing the library to be assayed with a receptor based assay rather than a toxin based assay.

We have also surprisingly found that the use of the A1 domain of the A chain is sufficient for performing the assays described herein. Although the A1 domain contains the entirety of the cytotoxic domain of the A subunit, it was not known until know what function was served by the A2 subunit in assays of this kind, and whether the A1 subunit could be successfully used on its own in this type of assay. By removing the A2 portion of the A chain, a smaller, more easily manipulated fragment can be used in the assays described herein.

Combining the unexpected finding that cysteine mutations result in a functional A subunit useful in the assays described herein, with the finding that the A1 subunit can be used alone yields the further advantage of combining these two findings. By mutating one of the cysteines (for example, C261), one can make a protein that can not form the protease-sensitive A chain loop (since this protease-sensitive A chain loop is a result of the cysteine-cysteine interactions between C242 and C261). The A subunit can therefore be cleaved more efficiently into A1 and A2 subunits. Thus mutation of at least one of C242 and C261, combined with utilizing the A1 subunit alone, provides a simpler and more elegant strategy for purifying single chain toxins comprising the A1 domain of A chain mutants. Manufacturing, and purifying, a protein or a protein library of use in the assays described herein thus becomes much more efficient by either utilizing the A1 domain alone, at least one cysteine mutation, or both.

A further aspect of the invention is a combinatorial expression library comprising a plurality of species of expression systems. Each species within the expression library expresses a protein species comprising an A or A1 chain of a heteromeric toxic protein into which an insert has been introduced. The insert is a polypeptide of varying amino acid sequence having a length of 2 or more amino acid residues, for example from 3 to 200 amino acid residues; and is introduced into the protease-sensitive loop of the A chain sequence. Suitable expression systems include plasmids and viral vectors.

In a further aspect of the invention, the A or A1 chain can be pegylated or otherwise modified by a method known in the art in order to stabilize or modify the biological half-life of the protein. Reactive pegylation agents can be bound to a lysine, to the N-terminus, or to cysteine 242 or 261.

We have also found that adding a histidine tag (for example, a 6 or 8 histidine tag (SEQ ID NOS 89-90, respectively) to the N-terminus of the A or A1 chain is advantageous as it allows for improved purification of the desired protein. Optionally, a protease-sensitive region can be inserted between the histidine tag and the rest of the A or A1 chain in order to facilitate cleavage of the histidine tag from the A or A1 chain once it is no longer desired. Examples of protease-sensitive regions that may be used include a thrombin sensitive region or a furin sensitive region. Other protease-sensitive sequences are also well known in the art. The invention will now be further described with reference to the following, non-limiting examples.

### Example 1

### Proof-of-Concept: Design and Mining of a Prototypic a Chain-Tripeptide Library

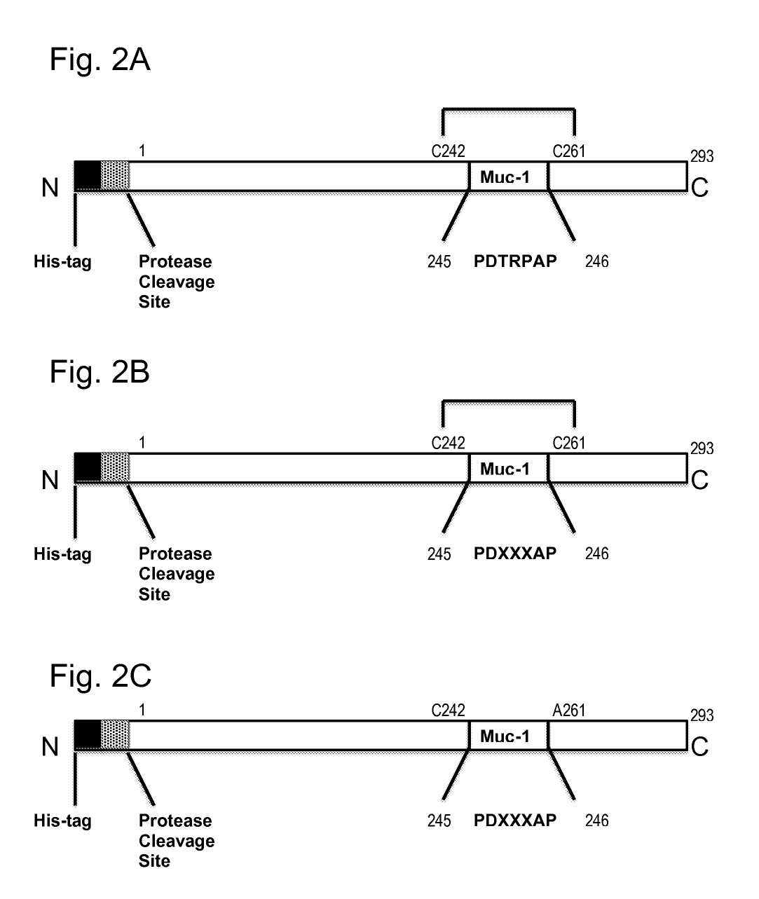

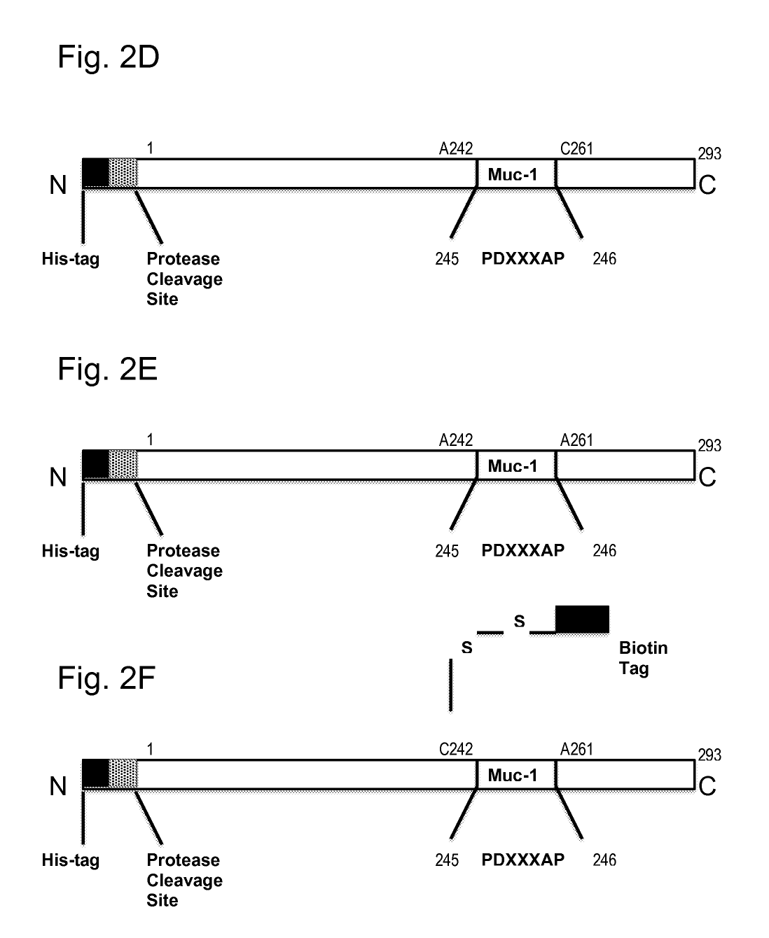

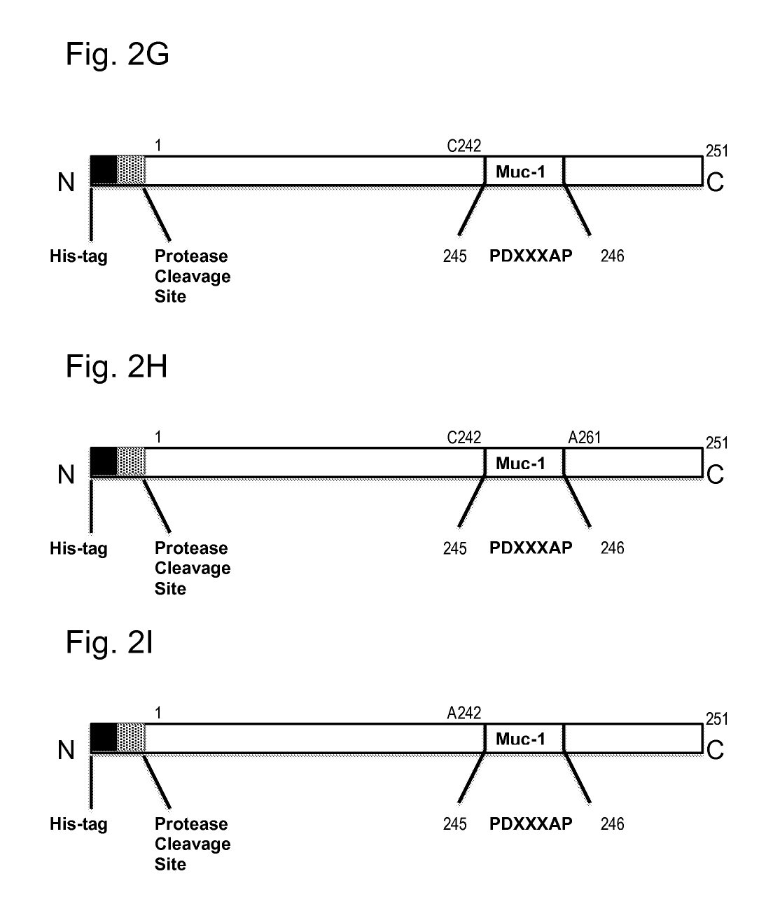

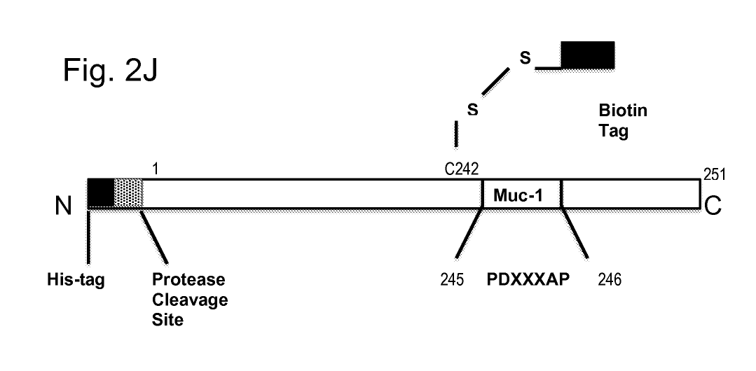

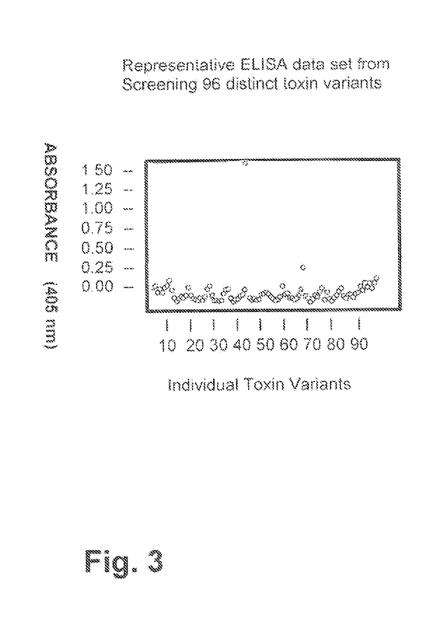

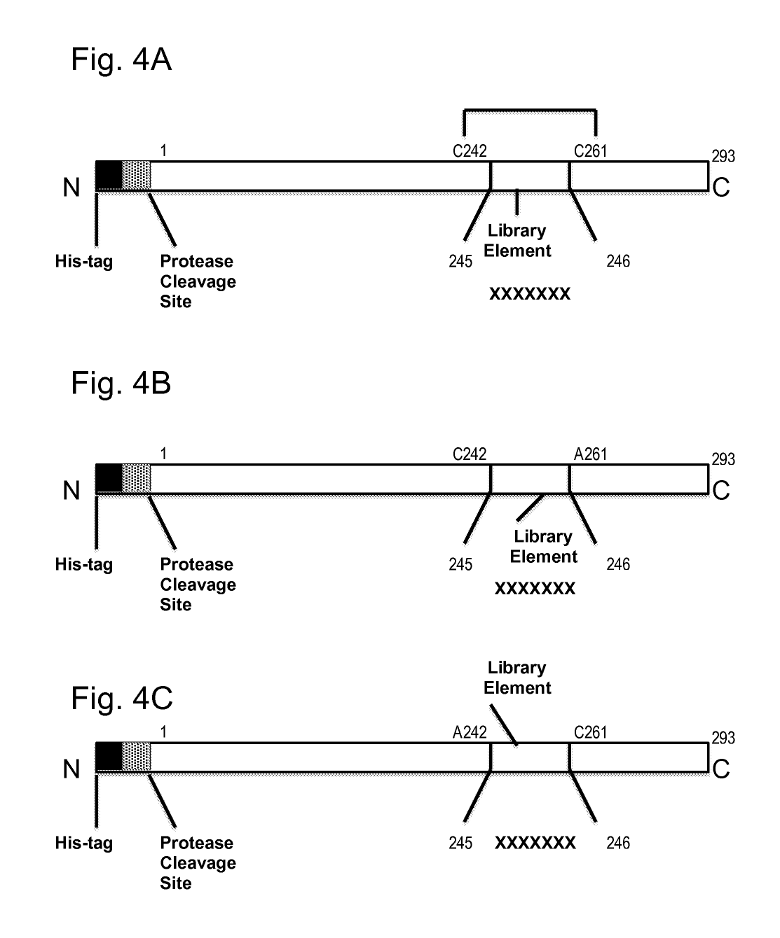

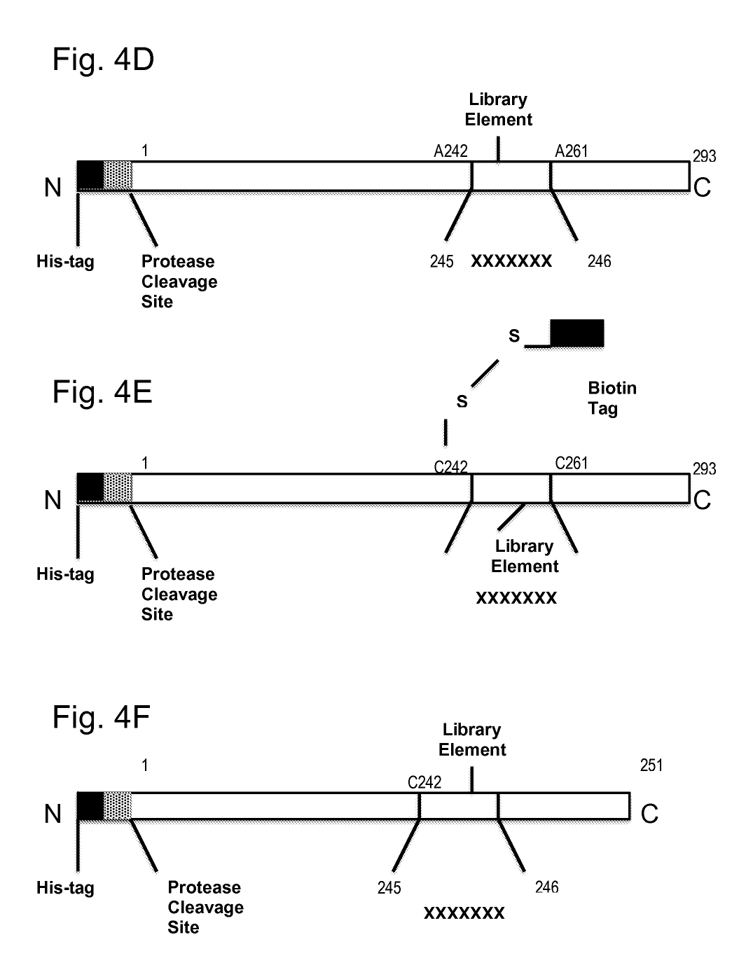

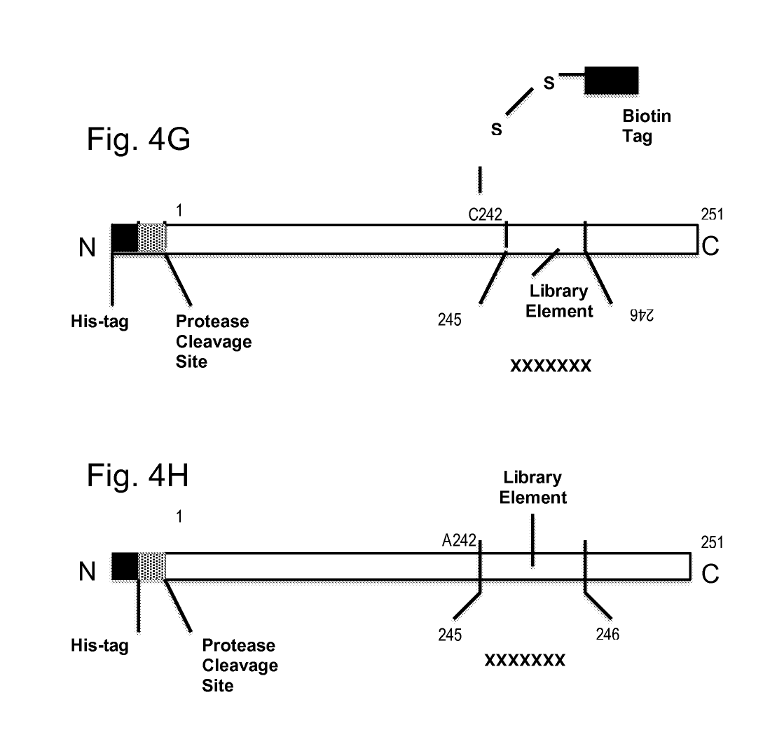

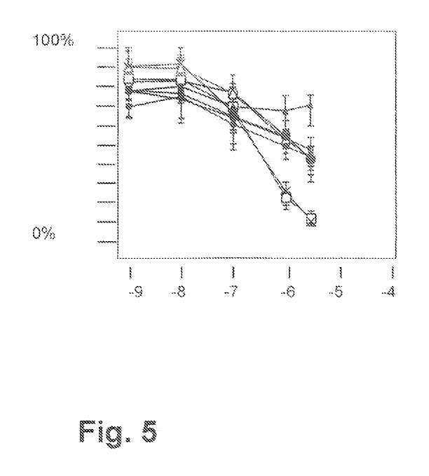

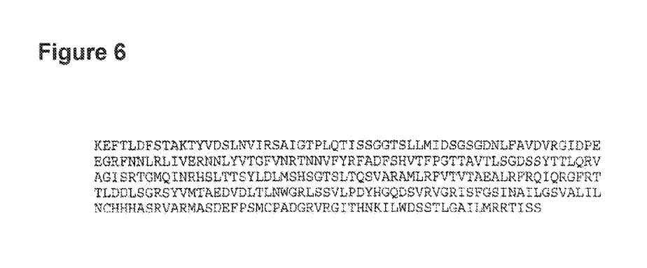

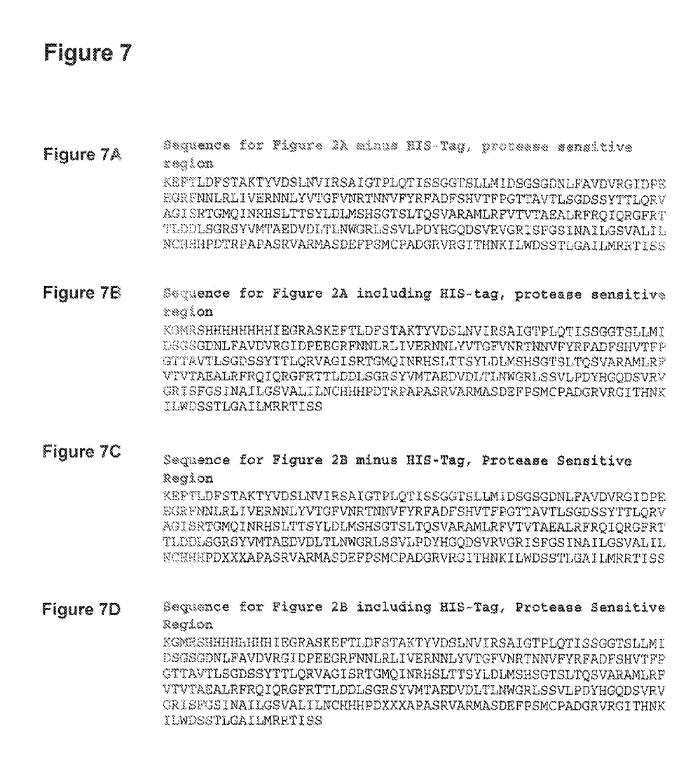

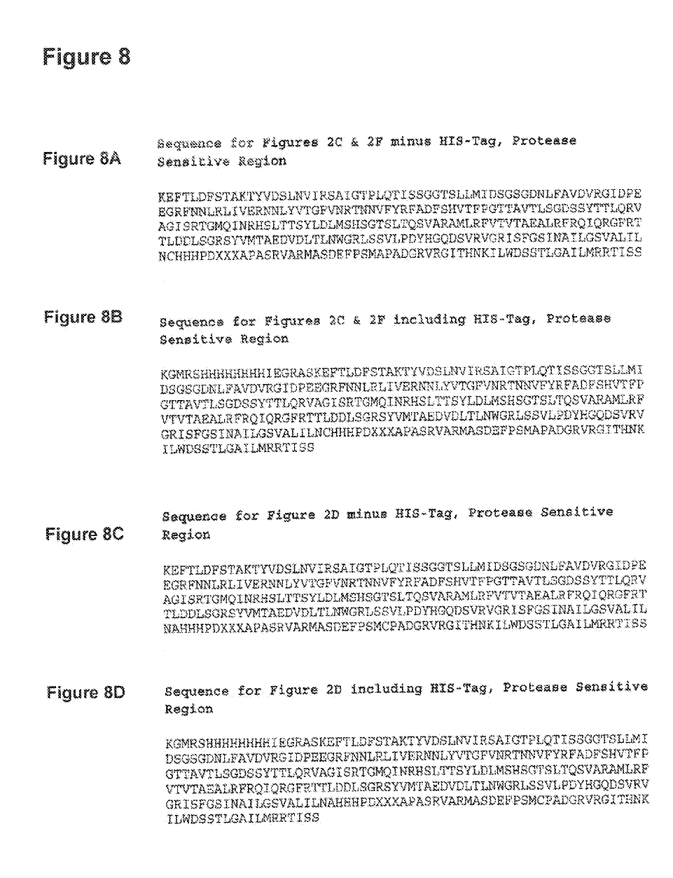

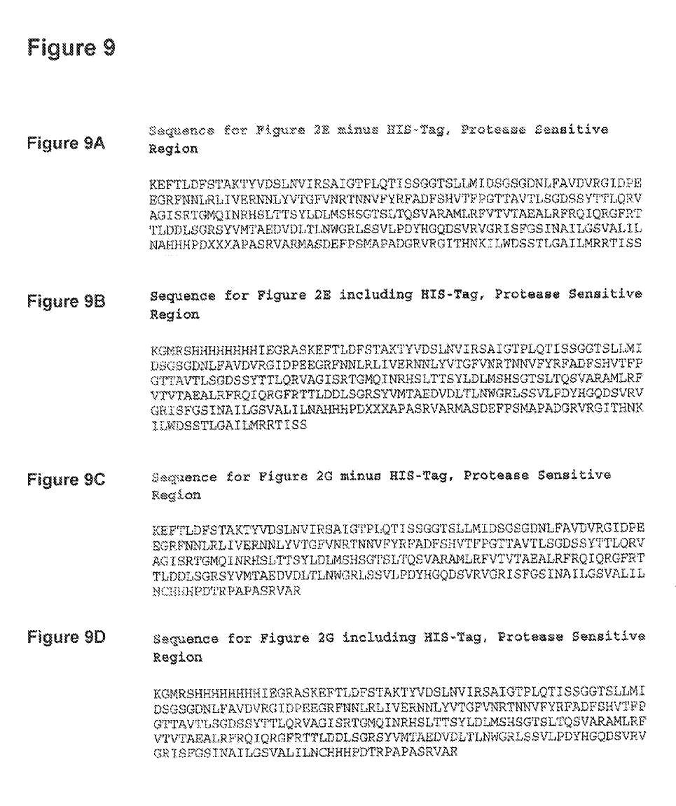

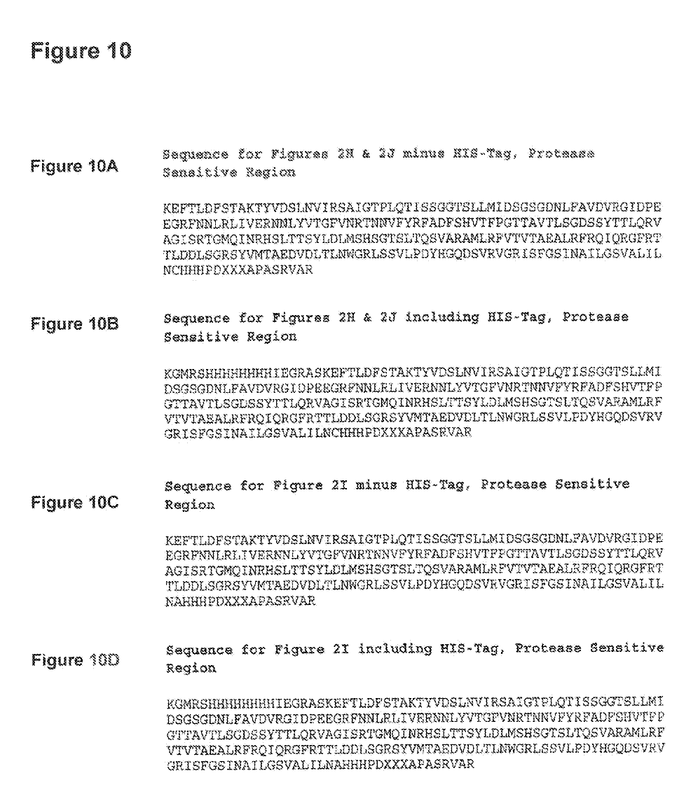

We originally created a simple tripeptide library inserted in the C-terminal pro tease-sensitive loop of the SLT-I A chain (FIGS. 2 A and B). This A chain loop region is naturally constrained due to the presence of a single disulfide bond bridging Cys242 to Cys261. The maximal diversity of this library can thus be calculated to be 20<3> or 8000 permutations of a tripeptide sequence. As a proof-of-concept that A chain libraries can easily be screened for a new receptor-binding activity, we picked more than 3000 colonies from this A chain-tripeptide library and purified the mutant toxin produced by each clone. We noticed very early in this study that the level of expression of A chain mutant was dramatically increased when expressed in the presence of the wild-type SLT-I B subunit. Thus the mutant forms of A chain were expressed and initially purified as AB5 toxin variants. Since all A subunits harbor a polyHis purification tag, it is relatively easy to remove the B subunit with denaturants (urea for example) while recovering the A chain on metal-affinity columns or beads. Western blots performed on randomly selected bacterial clones indicated that >70% of these colonies produced significant amounts of these A chain mutants.

These toxin variants were then coated in individual wells of 96-well plates and screened by ELISA for their ability to bind the monoclonal antibody One M27 (Linsley, et al. (1988) Monoclonal antibodies reactive with mucin glycoproteins found in sera from breast cancer patients. Cancer Res 48, 2138-2148.), directed at the well-characterized breast cancer tripeptide epitope Thr-Arg-Pro of the human MUCl tandem repeat (Gendler, et al. (1988) A highly immunogenic region of a human polymorphic epithelial mucin expressed by carcinomas is made up of tandem repeats. J Biol Chem 263, 12820-12823; Girling, et al. (1989) A core protein epitope of the polymorphic epithelial mucin detected by the monoclonal antibody SM-3 is selectively exposed in a range of primary carcinomas. Int J Cancer 43, 1072-1076). As shown in Tables 1 and 2, most A chain mutants neither coded for the tripeptide insert that matched the targeted epitope of One M27 nor were recognized by the antibody. However on two occasions, a toxin variant harboring the exact epitope (Table 1, FIG. 3) gave a strong ELISA signal comparable to the one observed with our control. A chain harboring the MUCl tripeptide epitope and had the expected epitope sequence in its randomized tripeptide region. A typical ELISA data set for 96 A chain mutants is presented in FIG. 3, highlighting the fact that the majority of A chain mutants did not recognized the mAb One M27 except for an A chain variant (mutant #41 in Tables 1 and 2). These results clearly established that A chain libraries can easily be constructed and screened to find toxin variants able to specifically target a given receptor, in this case an antigen-combining site.

### Example 2

### Proof-of-Concept: Design and Mining of a Prototypic Chain-Tripeptide Library

A tripeptide library inserted in the C-terminal SLT-I A1 domain is prepared. A simple tripeptide library is inserted in the C-terminal pro tease-sensitive loop of the SLT-I A chain (FIGS. 2A and B). This A chain loop region is naturally constrained due to the presence of a single disulfide bond bridging Cys242 to Cys261. The maximal diversity of this library can thus be calculated to be 20<3> or 8000 permutations of a tripeptide sequence. A chain libraries can easily be screened for a new receptor-binding activity. Over 3000 colonies from this A chain tripeptide library are picked and the mutant toxin produced by each clone is purified. The level of expression of A chain mutant is dramatically increased when expressed in the presence of the wild-type SLT-I B subunit. Thus the mutant forms of A chain are expressed and initially purified as AB5 toxin variants. Since all A subunits harbor a polyHis purification tag, it is relatively easy to remove the B subunit with denaturants (urea for example) while recovering the A chain on metal-affinity columns or beads. Western blots performed on randomly selected bacterial clones indicate that >70% of these colonies produce significant amounts of these A chain mutants. The A1 subunit is then excised from the A chain mutants, using a protease cleavage using furin (FIGS. 2G and H).

These toxin variants are then coated in individual wells of 96-well plates and screened by ELISA for their ability to bind the monoclonal antibody One M27 (Linsley, et al. (1988) monoclonal antibodies reactive with mucin glycoproteins found in sera from breast cancer patients. Cancer Res 48, 2138-2148.), directed at the well-characterized breast cancer tripeptide epitope Thr-Arg-Pro of the human MUCl tandem repeat (Gendler, et al. (1988) A highly immunogenic region of a human polymorphic epithelial mucin expressed by carcinomas is made up of tandem repeats. J Biol Chem 263, 12820-12823; Girling, et al. (1989) A core protein epitope of the polymorphic epithelial mucin detected by the monoclonal antibody SM-3 is selectively exposed in a range of primary carcinomas. Int J Cancer 43, 1072-1076). Most A1 chain mutants neither code for the tripeptide insert that match the targeted epitope of One M27 nor are recognized by the antibody. However on a few occasions, a toxin variant harboring the exact epitope gives a strong ELISA signal comparable to the one observed with our control A chain harboring the MUCl tripeptide epitope and has the expected epitope sequence in its randomized tripeptide region. These results clearly establish that A1 chain libraries can easily be constructed and screened to find toxin variants able to specifically target a given receptor, in this case an antigen-combining site.

### Example 3

### Proof-of-Concept: Alternative Method for Design and Mining of a Prototypic A1 Chain-Tripeptide Library

A tripeptide library inserted in the C-terminal SLT-I A1 domain is prepared. A simple tripeptide library is inserted in the C-terminal protease-sensitive loop of the SLT-I A1 chain (FIGS. 2 G and H). This A1 domain codes for A region amino acids 1-251. The maximal diversity of this library can thus be calculated to be 20<3> or 8000 permutations of a tripeptide sequence. A chain libraries can easily be screened for a new receptor-binding activity. Over 3000 colonies from this A1 chain tripeptide library are picked and the mutant toxin produced by each clone is purified. Since all A1 subunits lack a B subunit binding area, it is relatively easy to exise any B subunits. Optionally, the polyHIS tags can be used to further purify the A1 subunits, as described in previous examples.

These toxin variants are then coated in individual wells of 96-well plates and screened by ELISA for their ability to bind the monoclonal antibody One M27 (Linsley, et al. (1988) monoclonal antibodies reactive with mucin glycoproteins found in sera from breast cancer patients. Cancer Res 48, 2138-2148.), directed at the well-characterized breast cancer tripeptide epitope Thr-Arg-Pro of the human MUCl tandem repeat (Gendler, et al. (1988) A highly immunogenic region of a human polymorphic epithelial mucin expressed by carcinomas is made up of tandem repeats. J Biol Chem 263, 12820-12823; Girling, et al. (1989) A core protein epitope of the polymorphic epithelial mucin detected by the monoclonal antibody SM-3 is selectively exposed in a range of primary carcinomas, hit J Cancer 43, 1072-1076). Most A1 chain mutants neither code for the tripeptide insert that match the targeted epitope of One M27 nor are recognized by the antibody. However on a few occasions, a toxin variant harboring the exact epitope gives a strong ELISA signal comparable to the one observed with our control A chain harboring the MUCl tripeptide epitope and has the expected epitope sequence in its randomized tripeptide region. These results clearly establish that A1 chain libraries can easily be constructed and screened to find toxin variants able to specifically target a given receptor, in this case an antigen-combining site.

The A1 tripeptide library can also be biotinylated at the C242 position (FIG. 2J). A standard biotin assay can then be used to determine whether an A1 tripeptide library member is present, or bound to a substrate or given receptor.

### Example 4

### Proof-of-Concept: Method for Design and Mining of a Prototypic a Chain-Tripeptide Library with a Mutation in C242 and/or C 261

A tripeptide library inserted in the C-terminal SLT-I A domain is prepared, using the method of Example 1 with the modification that encoding for the cysteine at position 242 and/or position 261 has been mutated to an alanine using site directed mutagenesis (FIG. 2C, 2D, or 2E). For libraries containing one cysteine in the A chain, the tripeptide library is optionally biotinylated (FIG. 2F). The A chain libraries can easily be screened for a new receptor-binding activity. Results indicate that these cys to ala modified A chain libraries are at least as effective as the wild-type libraries of Example 1 in ease of construction and screening to find toxin variants able to specifically target a given receptor. In the case of the biotinylated libraries, the biotin labels are easily used, utilizing known methods, to identify whether a member of the A chain library is present or bound to a sample.

### Example 5

### Proof-of-Concept: Method for Design and Mining of a Prototypic A1 Chain-Tripeptide Library with a Mutation in C242 and/or C 261

A tripeptide library inserted in the C-terminal SLT-I A1 domain is prepared, using the method of Example 2 with the modification that encoding for the cysteine at position 242 and/or position 261 has been mutated to an alanine using site directed mutagenesis (FIG. 21). The A1 chain libraries can easily be screened for a new receptor-binding activity. Results indicate that these cys to ala modified A1 chain libraries are as effective as the wild-type libraries of Example 2 in ease of construction and screening to find toxin variants able to specifically target a given receptor. Results indicate that these cys to ala modified A1 chain libraries are significantly easier to make and purify, due to the lack of the disulfide bridge between Cys 242 and Cys 261, resulting in a much easier cleavage of A2 from A1 in the protease cleavage step. In the case of an A1 tripeptide library having a cysteine at position C242, the cysteine is optionally tagged with biotin (FIG. 2J). This allows for determination of whether the library member is present, or bound to a substrate or receptor, using known methods.

### Example 6

### Proof-of-Concept: Method for Design and Mining of a Prototypic A1 Chain-Tripeptide Library with a Mutation in C242 and/or C 261

A tripeptide library inserted in the C-terminal SLT-I A1 domain is prepared, using the method of Example 3 with the modification that encoding for the cysteine at position 242 has been mutated to an alanine using site directed mutagenesis (FIG. 21). The A1 chain libraries can easily be screened for a new receptor-binding activity. Results indicate that these cys to ala modified A1 chain libraries are as effective as the wild-type libraries of Example 4 in ease of construction and screening to find variants able to specifically target a given receptor.

### Example 7

### Making of Combinatorial SLT-I A-Heptapeptide or A1-Heptapeptide Library

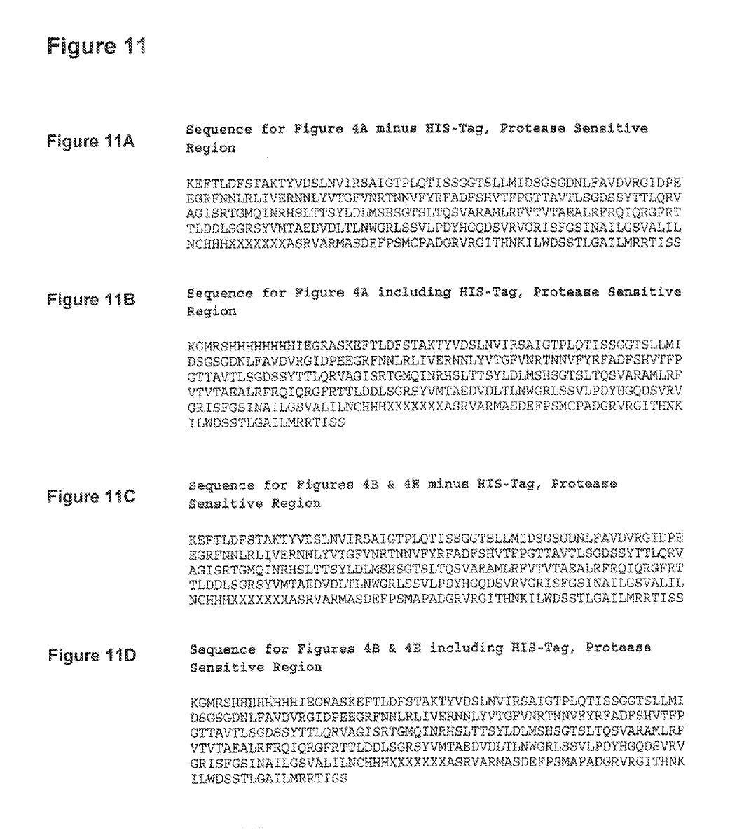

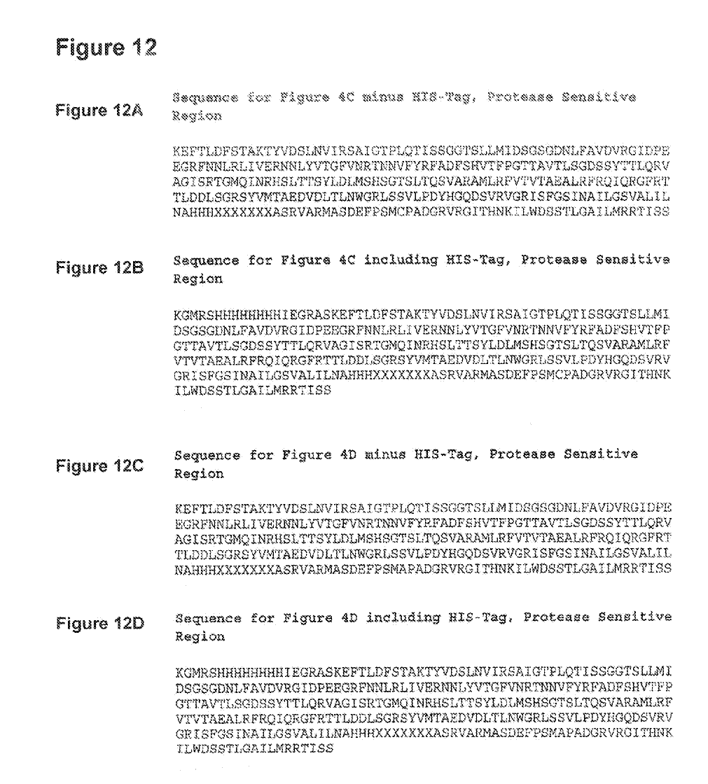

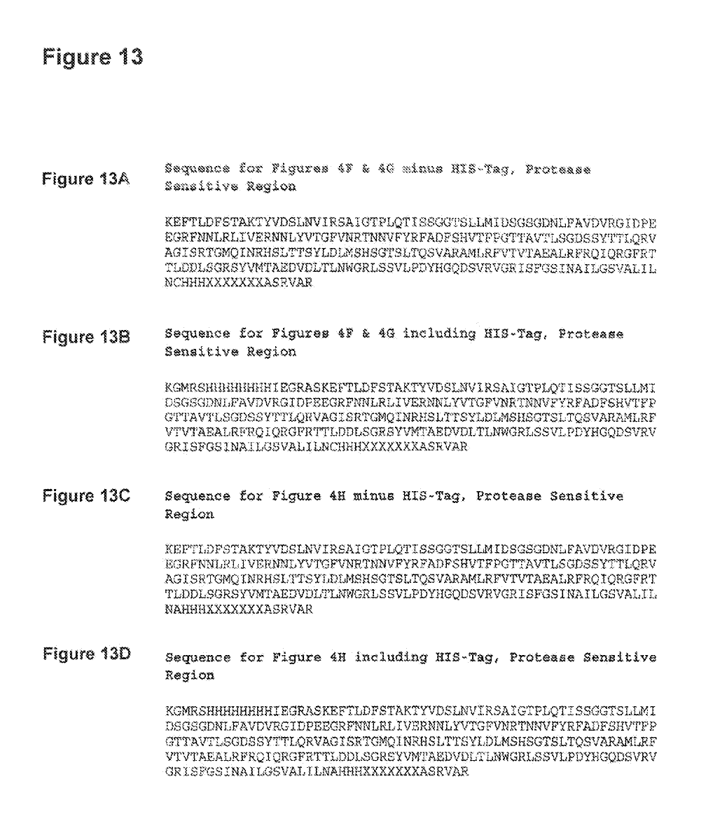

Library diversity represents a crucial parameter in screening combinatorial libraries for ligands able to bind specifically and with high affinity to a particular target. The SLT-I A chain-tripeptide library described in Examples 1 or 4, and the SLT-I A1 chain-tripeptide libraries described in Examples 2, 3, 5 and 6 have a maximal diversity of 20<3> or 8000 possible mutated A chains. This sampling repertoire is small but was useful in mapping a tripeptide epitope to establish our proof-of-concept. A seven-residue library (20<7> or 1.3×10<9> possible mutants) represents a more typical minimal diversity level commonly used in designing phage display as well as synthetic peptide libraries. Thus, as a starting point, we build SLF-I A and A1 chain libraries as described in Examples 1-6) with a 7-amino acid long random sequence inserted in its C-terminus (FIG. 4 A-H). Each of these libraries provide sufficient diversity to insure that A chain or A1 chain toxin variants can be identified that target new or known internalized receptors on cancer cells. All elements of each of these libraries (as well as all other libraries proposed) contain a N-terminal His tag to quickly purify A chain mutants. The libraries (FIG. 4A-4H) are generated using a megaprimer PCR strategy (Sarkar G, and Sommers S, (1990). The ‘megaprimer’ method of site-directed mutagenesis. Biotechniques 8, 404-407). The megaprimer strategy is widely used to introduce mutations into target DNA sequence, by way of two rounds of PCR that use two flanking primers and an internal mutagenic primer.

All A chain-heptapeptide libraries and A1 chain-heptapeptide libraries are made, as follows. The SLT-I A chain-heptapeptide libraries (FIG. 4A-4H) harbor a 7-amino acid random insertion between amino acid 245 and 246 of the A or A1 chain, the identical site used to construct our SLT-I A chain-tripeptide library (Tables 1, 2, FIG. 3). Briefly, construction of the A chain heptapeptide libraries is described as follows using as the example an A chain containing 2 cysteines. Construction of the A1 chain heptapeptide libraries is undertaken using similar methods, with modifications as necessary to form an A1 chain (and as described in parallel Examples 2, 3, 5 and 6, above).

Two flanking primers A (GTT ACT GTG ACA GCT GAA GCT TTA CGT TTT CG (Seq. ID No. 2) and B (GAG AAG AAG AGA CTG CAG ATT CCA TCT GTT G (Seq. ID No. 3)) carrying Hindi 11 and Pstl restriction sites respectively were annealed within the 5′ and 3′ ends of the SLT-I operon. A library oligonucleotide F containing all seven random amino acid (NNS) as well as a long matching sequence to anneal to the template were synthesized. In the synthesis of the random oligonucleotide, the relative representation of each amino acid was improved by restricting the third position of each codon to G or T (Noren, K. A., and Noren, C. J. (2001) Construction of high-complexity combinatorial phage display peptide libraries. Methods 23, 169478.). This type of restriction reduces the overall DNA sequence complexity as well as coding discrepancy between residues (Reidhaar-Olson, et al. (1991) Random mutagenesis of protein sequences using oligonucleotide cassettes. Methods Enzymol 208, 564-586.). This strategy also minimizes the occurrence of stop codons (TAA and TGA) while the stop codon (TAG) is suppressed by using a supE bacterial strain, which specifies for the insertion of a Gln residue when TAG codons are translated. The first PCR reaction was performed using primers A and F and the resulting product purified by denaturing polyacrylamide gel electrophoresis. This product then served as the megaprimer with primer B for a second PCR reaction to amplify the random DNA. The final library DNA (PCR product) will then be digested with Hindi 11 and Pstl and cloned into the backbone of a pECHE9a expression vector (MTI, Toronto). The E. coli strain JMlOl was subsequently transformed with the resulting pECHE vector and single bacterial colonies picked, lysed and their supernatants analyzed for the expression of single A chain toxins or layered on cancer cells and screened using the SRB for cell cytotoxicity assay.

### Example 8

### Mining the Combinatorial SLT-I A-Heptapeptide Library Against Cancer Cell Lines Using a Cytotoxicity Assay

We screen our SLT-I A-heptapeptide libraries and our SLT-I A1-heptapeptide libraries using the cytotoxic function of the A or A1 chain as a reporter signal. Cytotoxicity is a more informative property to measure than binding to a receptor since it implies that the toxin is internalized, processed and delivered near ribosomes, clearly a multi-step event. The cytotoxicity assay is essentially performed as previously described (Bray, et al. (2001) Probing the surface of eukaryotic cells using combinatorial toxin libraries. Current Biology 11, 697-701). Briefly, the strategy to screen all our A chain libraries is based on the following principles. Established cancer cell lines such as SK-BR-3 (human breast), CAMA-I (human breast), 518A2 (human melanoma), PC3 (human prostate) and B16 (murine melanoma) are grown in 96-well plates and used as targets in the primary screen stages. These cell lines are initially selected for our holotoxin library searches (Bray, supra) based on their adherence (plastic), their cell viability staining properties (SRB) in a high-throughput screening setting as well as their lack of receptor and sensitivity to native SLT-I (to insure a reduced level of false positives). Single bacterial colonies from each library are picked and grown in 96 deep well plates. The cells are harvested, lysed, and their lysates clarified. Since all expressed SLT-I A chain variants or A1 chain variants have a histidine tag at their N-terminus, each of them is purified from their lysate using nickel-affinity beads (96-well format) and layered on target cells. The plates containing the target cells treated with A chain variants or A1 chain variants are then incubated at 37[deg.]C. for 48 hours, followed by fixation and staining with Sulforhodamine B (SRB). The SRB assay is a colorimetric end-point assay, which quantifies viable cells by staining their cellular protein content (Skehan, et al. (1990) New colorimetric cytotoxicity assay for anticancer-drug screening. J Natl Cancer hist 82, 1107-1112.). The SRB assay has been adopted by NCl/NIH for their high-throughput screening of drug candidates on cancer cell lines. Viability assays are repeated for any bacterial extracts leading to cell death. A first round of screening is performed on more than 5000 bacterial clones (equivalent to 5000 distinct A chain toxins) for each of the A chain and A1 chain pentapeptide libraries as described in FIGS. 4A to 4H, and several toxin variants are identified in each library as repeatable killers of the human melanoma cell line 518A2 (FIG. 5). We find that the A1 chain libraries of FIGS. 4 F, 4 G, and 4 H have similar cytotoxic properties as of that of the A chain libraries. We also find that the libraries with mutations at cysteine 242 or cysteine 261, or at both cysteines, as described in FIGS. 4B, 4C, 4D, and 4E also have similar cytotoxic properties as that of the “wild type” A chain library of FIG. 4A.

The abscissa represents the log concentration of toxin used to treat the cells and the ordinate depicts the observed percentage of cells that are viable after 48 hours. The closed triangles depicts the effect of the wild type toxin on 518A2 cells while the two most efficacious A chain variants were termed SAM#3 (open squares) and SAM#5 (X symbols).

Promising A chain and A1 chain variants from each of the heptapeptide libraries are then re-screened against a panel of cell lines (Vero [Monkey, normal kidney]; PC-3 [Human, prostate cancer]; HepG2 [Human, hepatoma]; SiHa [Human, cervical cancer]; PanC [Human, pancreatic cancer]; SKBR-3 [Human, breast cancer]; 518-A2 [Human, melanoma]; U87 [Human, glioma]; 1316-F10 [Mouse, melanoma]; HS-216 [Human, normal fibroblast]; CAMA-1 [Human, breast cancer]; OVCar-3 [Human, ovarian cancer]). Of these variants, a significant proportion are observed to have activity against each of the cancer cell line, the 518-A2 human melanoma, and the SiHa (human cervical cancer cells) and U87-A (human brain cancer cells; glioma) cell lines.

The genes coding for the two A chain toxins (SAM3 and SAM5) that resulted in toxicity of the human melanoma cell lines were sequenced to determine the amino acid sequences inserted between residues 245 and 246 of the wild-type A chain. The sequences, including the His-tag, are listed in Seq. ID Nos 4 and 5, respectively.

TABLE 1
DNA sequences (SEQ ID NOS 65-75, respectively, in
order of appearance) of randomly picked clones
from the SLT-I A chain-tripeptide library.

| | | Diversity |
|---|---|---|
| | | (nucleotide |
| | | changes in |
| SLT-1 A | | mutated |
| chain | Nucleotide sequence | region) |
| MUCl | CCA GAC ACG CGA CCA GCT CCA | 0/9 |
| epitope | | |
| Mutant #1 | CCA GAC GGG ATC GGG GCT CCA | 8/9 |
| Mutant #2 | CCA GAC CTG GAG ATG GCT CCA | 8/9 |
| Mutant #3 | CCA GAC CCC CGT GGG GCT CCA | 6/9 |
| Mutant #4 | CCA GAC GAC GAC TTG GCT CCA | 9/9 |
| Mutant #5 | CCA GAC GTC CGG TGG GCT CCA | 7/9 |
| Mutant #6 | CCA GAC CAG CGC TGG GCT CCA | 6/9 |
| Mutant #7 | CCA GAC CTC AGG ATG GCT CCA | 8/9 |
| Mutant #8 | CCA GAC TCC CAG GAG GCT CCA | 7/9 |
| Mutant #9 | CCA GAC TCC GAC CCC GCT CCA | 6/9 |
| Mutant #41 | CCA GAC ACG CGC CCC GCT CCA | 2/9 |

Mutated bases in bold characters.
Mutant #41 was identified in our ELISA screen as a strong binder of mAb One M27 (FIG. 3).

TABLE 2
Amino acid sequence (SEQ ID NOS 76-86,
respectively, in order of appearance) alignment
of randomly selected clones from the SLT-I A
chain-tripeptide library and ELISA signal of
purified SLT-I A chain variants detected with a
rnAb (One M27) raised against the MUCl epitope
Thr-Arg-Pro.

| | | ELISA |
|---|---|---|
| | | readings |
| SLT-I | | variant |
| A chain | Deduced amino acid sequence | (405 nm) |
| MUCl epitope | CHHHPD TRP APASRVARMASDEFPSMC | 1.3 |
| Mutant #1 | CHHHPD GIG APASRVARMASDEFPSMC | 0.08 |
| Mutant #2 | CHHHPD LQM APASRVARMASDEFPSMC | 0.03 |
| Mutant #3 | CHHHPD PRG APASRVARMASDEFPSMC | 0.03 |
| Mutant #4 | CHHHPD DDL APASRVARMASDEFPSMC | 0.06 |
| Mutant #5 | CHHHPD VRW APASRVARMASDEFPSMC | 0.07 |
| Mutant #6 | CHHHPD QRL APASRVARMASDEFPSMC | 0.06 |
| Mutant #7 | CHHHPD LRM APASRVARMASDEFPSMC | 0.11 |
| Mutant #8 | CHHHPD SQE APASRVARMASDEFPSMC | 0.13 |
| Mutant #9 | CHHHPD SDP APASRVARMASDEFPSMC | 0.07 |
| Mutant #41 | CHHHPD TRP APASRVARMASDEFPSMC | 1.25 |

Mutated tripeptide region in bold characters.
Mutant #41 was identified in our ELISA screen as a strong binder of mAb One M27.

We claim:

1. A mutant protein comprising a modified A chain of a toxic protein into which an insert has been introduced wherein the insert comprises SEQ ID NO:6.

2. The mutant protein of claim 1 wherein the insert is introduced in a protease-sensitive loop of the wild-type A chain sequence.

3. The mutant protein of claim 1 wherein the modified A chain contains a mutation of at least one cysteine.

4. The mutant protein of claim 3 wherein the mutation of at least one cysteine is a mutation from cysteine to alanine.

5. The mutant protein of claim 1 wherein the wild-type A chain of a toxic protein is a Shiga-like toxin I A chain.

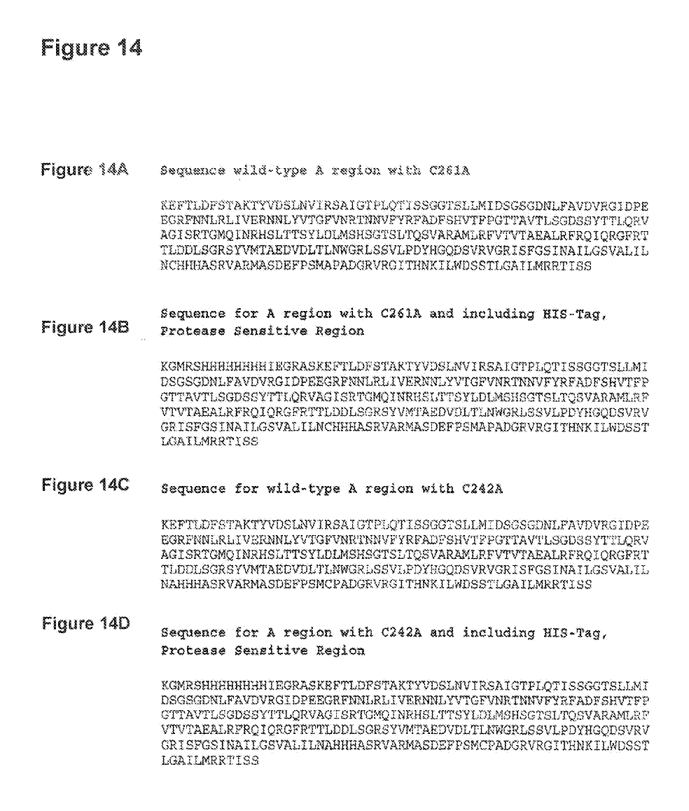

6. The mutant protein of claim 5 wherein the insert is introduced between amino acids 242 and 261 as defined with reference to the sequences of FIG. 14A, 14C, or 15A.

7. The mutant protein of claim 5 wherein the insert is introduced between amino acids 245 and 246 as defined with reference to the sequences of FIG. 14A, 14C, or 15A.

8. The mutant protein of claim 5 wherein the insert is introduced before or after amino acids 1-239 as defined with reference to the sequences of FIG. 14A, 14C or 15A.

9. The mutant protein of claim 1 comprising the polypeptide sequence of SEQ ID NO:4.

10. The mutant protein of claim 1 wherein the protein has been pegylated.

11. A mutant protein comprising a modified A chain of a toxic protein into which an insert has been introduced wherein the insert comprises SEQ ID NO:7.

12. The mutant protein of claim 11 wherein the insert is introduced in a protease-sensitive loop of the wild-type A chain sequence.

13. The mutant protein of claim 11 wherein the modified A chain contains a mutation of at least one cysteine.

14. The mutant protein of claim 13 wherein the mutation of at least one cysteine is a mutation from cysteine to alanine.

15. The mutant protein of claim 11 wherein the wild-type A chain of a toxic protein is a Shiga-like toxin I A chain.

16. The mutant protein of claim 15 wherein the insert is introduced between amino acids 242 and 261 as defined with reference to the sequences of FIG. 14A, 14C, or 15A.

17. The mutant protein of claim 15 wherein the insert is introduced between amino acids 245 and 246 as defined with reference to the sequences of FIG. 14A, 14C, or 15A.

18. The mutant protein of claim 15 wherein the insert is introduced before or after amino acids 1-239 as defined with reference to the sequences of FIG. 14A, 14C or 15A.

19. The mutant protein of claim 11 comprising the polypeptide sequence of SEQ ID NO:5.

20. The mutant protein of claim 11 wherein the protein has been pegylated.

21. A method of treating cancer said method comprising the step of administering the mutant protein of claim 1 to a patient in need of such treatment wherein the cancer is selected from the group consisting of melanoma, cervical cancer, and brain cancer.

22. A method of treating cancer said method comprising the step of administering the mutant protein of claim 11 to a patient in need of such treatment wherein the cancer is selected from the group consisting of melanoma, cervical cancer, and brain cancer.


SEQUENCE LISTING (96 sequences)

SEQ ID NO: 1 - protein, 293 aa, Artificial Sequence
  Description of Artificial Sequence: Synthetic polypeptide
  Shiga-like toxin
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 2 - DNA, 32 bp, Artificial Sequence
  Description of Artificial Sequence: Synthetic primer A
gttactgtgacagctgaagctttacgttttcg

SEQ ID NO: 3 - DNA, 31 bp, Artificial Sequence
  Description of Artificial Sequence: Synthetic primer B
gagaagaagagactgcagattccatctgttg

SEQ ID NO: 4-5 - skipped (2 entries)

SEQ ID NO: 6 - protein, 7 aa, Artificial Sequence
  Description of Artificial Sequence: Synthetic peptide
  Mutant Shiga-like toxin protein
IYSNKLM

SEQ ID NO: 7 - protein, 7 aa, Artificial Sequence
  Description of Artificial Sequence: Synthetic peptide
  Mutant Shiga-like toxin protein
AAFADLI

SEQ ID NO: 8 - protein, 300 aa, Artificial Sequence
  Description of Artificial Sequence: Synthetic polypeptide
  Shiga-like toxin 1 A with the breast cancer-associated MUC1 epitope PDTRPAP less the N-terminus tags
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHPDTRPAPASRVARMASDEFPSMCPADGRVRGITHNKILWDSSTLGAILMRRTISS

SEQ ID NO: 9 - protein, 319 aa, Artificial Sequence
  Description of Artificial Sequence: Synthetic polypeptide
  Shiga-like toxin with the breast cancer-associated MUC1 epitope PDTRPAP with an example of one possible N-terminus tag
KGMRSHHHHHHHHIEGRASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHPDTRPAPASRVARMASDEFPSMCPADGRVRGITHNKILWDSSTLGAILMRRTISS

SEQ ID NO: 10 - protein, 319 aa, Artificial Sequence
  Description of Artificial Sequence: Synthetic polypeptide
  Shiga-like toxin 1 A chain with the breast cancer-associated MUC1 epitope PDTRPAP with an example of one possible N-terminus
KGMRSHHHHHHHHRVARASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHPDTRPAPASRVARMASDEFPSMCPADGRVRGITHNKILWDSSTLGAILMRRTISS

SEQ ID NO: 11 - protein, 300 aa, Artificial Sequence
  Description of Artificial Sequence: Synthetic polypeptide
  SLT-1 A chain-tripeptide library construction where the three positions of the MUC1 epitope recognized by the mAb Onc M27 are randomized less the N-terminus
  MOD_RES (248)..(250): Any amino acid
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHPDXXXAPASRVARMASDEFPSMCPADGRVRGITHNKILWDSSTLGAILMRRTISS

SEQ ID NO: 12 - protein, 319 aa, Artificial Sequence
  Description of Artificial Sequence: Synthetic polypeptide
  SLT-1A chain-tripeptide library construction where the three positions of the MUC1 epitope recognized by the mAb Onc M27 are randomized with an example of one possible N-terminus tag
  MOD_RES (267)..(269): Any amino acid
KGMRSHHHHHHHHIEGRASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHPDXXXAPASRVARMASDEFPSMCPADGRVRGITHNKILWDSSTLGAILMRRTISS

SEQ ID NO: 13 - protein, 319 aa, Artificial Sequence
  Description of Artificial Sequence: Synthetic polypeptide
  SLT-A chain tripeptide library construction where the three psotions of the MUC 1 epitope recognized by the mAb Onc M27 are randomized with an example of one possible N-terminus tag
  MOD_RES (267)..(269): Any amino acid
KGMRSHHHHHHHHRVARASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHPDXXXAPASRVARMASDEFPSMCPADGRVRGITHNKILWDSSTLGAILMRRTISS

SEQ ID NO: 14 - protein, 300 aa, Artificial Sequence
  Description of Artificial Sequence: Synthetic polypeptide
  SLT-1A chain tripeptide library wherein cysteine 261 has been mutated to an alanine and where the C242 residue has been biotinylated less the N-terminus tag
  MOD_RES (248)..(250): Any amino acid
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHPDXXXAPASRVARMASDEFPSMAPADGRVRGITHNKILWDSSTLGAILMRRTISS

SEQ ID NO: 15 - protein, 319 aa, Artificial Sequence
  Description of Artificial Sequence: Synthetic polypeptide
  SLT-1A chain tripeptide library wherein cysteine 261 has been mutated to an alanine and where the C242 residue has been biotinylated with an example of one possible N-terminus tag
  MOD_RES (267)..(269): Any amino acid
KGMRSHHHHHHHHIEGRASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARANLRFVTVTAEALRFRQIQRGFRTTLDDLSGRSYVMTAEDVDLTLNWGRLSSVLPDYHGQDSVRVGRISFGSINAILGSVALILNCHHHPDXXXAPASRVARMASDEFPSMAPADGRVRGITHNKILWDSSTLGAILMRRTISS

SEQ ID NO: 16 - protein, 319 aa, Artificial Sequence
  Description of Artificial Sequence: Synthetic polypeptide
  SLT-1A chain tripeptide library wherein cysteine 261 has been mutated to an alanine and where the C242 residue has been biotinylated with an example of one possible N-terminus tag
  MOD_RES (267)..(269): Any amino acid
KGMRSHHHHHHHHRVARASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARANLRFVTVTAEALRFRQIQRGFRTTLDDLSGRSYVMTAEDVDLTLNWGRLSSVLPDYHGQDSVRVGRISFGSINAILGSVALILNCHHHPDXXXAPASRVARMASDEFPSMAPADGRVRGITHNKILWDSSTLGAILMRRTISS

SEQ ID NO: 17 - protein, 300 aa, Artificial Sequence
  Description of Artificial Sequence: Synthetic polypeptide
  SLT - 1A chain tripeptide library construction wherein cysteine 242 has been mutated to an alanine less an N-terminus tags
  MOD_RES (248)..(250): Any amino acid
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHPDXXXAPASRVARMASDEFPSMCPADGRVRGITHNKILWDSSTLGAILMRRTISS

SEQ ID NO: 18 - protein, 319 aa, Artificial Sequence
  Description of Artificial Sequence: Synthetic polypeptide
  SLT -1 A chain tripeptide library construction wherein cysteine 242 has been mutated to an alanine with an example of one possible N-terminus tag
  MOD_RES (267)..(269): Any amino acid
KGMRSHHHHHHHHIEGRASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHPDXXXAPASRVARMASDEFPSMCPADGRVRGITHNKILWDSSTLGAILMRRTISS

SEQ ID NO: 19 - protein, 319 aa, Artificial Sequence
  Description of Artificial Sequence: Synthetic polypeptide
  SLT -1 A chain tripeptide library construction wherein cysteine 242 has been mutated to an alanine with an example of one possible N-terminus tag
  MOD_RES (267)..(269): Any amino acid
KGMRSHHHHHHHHRVARASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHPDXXXAPASRVARMASDEFPSMCPADGRVRGITHNKILWDSSTLGAILMRRTISS

SEQ ID NO: 20 - protein, 300 aa, Artificial Sequence
  Description of Artificial Sequence: Synthetic polypeptide
  SLT- 1 A chain tripeptide library construction wherein cysteine 242 and cysteine 261 have both been mutated to alanine residues less the N-terminus tag
  MOD_RES (248)..(250): Any amino acid
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHPDXXXAPASRVARMASDEFPSMAPADGRVRGITHNKILWDSSTLGAILMRRTISS

SEQ ID NO: 21 - protein, 319 aa, Artificial Sequence
  Description of Artificial Sequence: Synthetic polypeptide
  SLT- 1 A chain tripeptide library construction wherein cysteine 242 and cysteine 261 have both been mutated to alanine residues with an exampel of one possible N-terminus tag
  MOD_RES (267)..(269): Any amino acid
KGMRSHHHHHHHHIEGRASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHPDXXXAPASRVARMASDEFPSMAPADGRVRGITHNKILWDSSTLGAILMRRTISS

SEQ ID NO: 22 - protein, 319 aa, Artificial Sequence
  Description of Artificial Sequence: Synthetic polypeptide
  SLT- 1 A chain tripeptide library construction wherein cysteine 242 and cysteine 261 have both been mutated to alanine residues with an example of one possible N-terminus tag
  MOD_RES (267)..(269): Any amino acid
KGMRSHHHHHHHHRVARASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHPDXXXAPASRVARMASDEFPSMAPADGRVRGITHNKILWDSSTLGAILMRRTISS

SEQ ID NO: 23 - protein, 258 aa, Artificial Sequence
  Description of Artificial Sequence: Synthetic polypeptide
  SLT-1 A1 chain with the breast cancer-associated MUC1 epitope PDTRPAP inserted between residues 245 and 246 and a Histidine tag followed by a protease cleavage site less a N-terminus tags
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHPDTRPAPASRVAR

SEQ ID NO: 24 - protein, 277 aa, Artificial Sequence
  Description of Artificial Sequence: Synthetic polypeptide
  SLT-1 A1 chain with the breast cancer-associated MUC1 epitope PDTRPAP inserted between residues 245 and 246 and a Histidine tag followed by a protease cleavage site with an example of one possible N-terminus tag
KGMRSHHHHHHHHIEGRASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHPDTRPAPASRVAR

SEQ ID NO: 25 - protein, 277 aa, Artificial Sequence
  Description of Artificial Sequence: Synthetic polypeptide
  SLT-1 A1 chain with the breast cancer-associated MUC1 epitope PDTRPAP inserted between residues 245 and 246 and a Histidine tag followed by a protease cleavage site with an example of one N-terminus tag
KGMRSHHHHHHHHRVARASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHPDTRPAPASRVAR

SEQ ID NO: 26 - protein, 258 aa, Artificial Sequence
  Description of Artificial Sequence: Synthetic polypeptide
  SLT-1 A1 chain tripeptide library construction where the three key positions of the MUC1 epitope recognized by the mAb Onc M27 were randomized and wherein cysteine 242 has been biotinylated less N-terminus tags
  MOD_RES (248)..(250): Any amino acid
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVNTAEDVDLTLNWGRLSSVLPDYHGQDSVRVGRISFGSINAILGSVALILNCHHHPDXXXAPASRVAR

SEQ ID NO: 27 - protein, 277 aa, Artificial Sequence
  Description of Artificial Sequence: Synthetic polypeptide
  SLT-1 A1 chain tripeptide library construction where the three key positions of the MUC1 epitope recognized by the mAb Onc M27 were randomized and wherein cysteine 242 has been biotinylated with an example of one possible N-terminus tag
  MOD_RES (267)..(269): Any amino acid
KGMRSHHHHHHHHIEGRASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGLSSYTTLQRVAGISRTGNQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHPDXXXAPASRVAR

SEQ ID NO: 28 - protein, 277 aa, Artificial Sequence
  Description of Artificial Sequence: Synthetic polypeptide
  SLT-1 A1 chain tripeptide library construction where the three key positions of the MUC1 epitope recognized by the mAb Onc M27 were randomized and wherein cysteine 242 has been biotinylated with an example of one possible N-terminus tag
  MOD_RES (267)..(269): Any amino acid
KGMRSHHHHHHHHRVARASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGLSSYTTLQRVAGISRTGNQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHPDXXXAPASRVAR

SEQ ID NO: 29 - protein, 258 aa, Artificial Sequence
  Description of Artificial Sequence: Synthetic polypeptide
  SLT-1 A1 chain tripeptide library construction wherein cysteine 242 has been mutated to an alanine less the N-terminus tag
  MOD_RES (248)..(250): Any amino acid
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVNTAEDVDLTLNWGRLSSVLPDYHGQDSVRVGRISFGSINAILGSVALILNAHHHPDXXXAPASRVAR

SEQ ID NO: 30 - protein, 277 aa, Artificial Sequence
  Description of Artificial Sequence: Synthetic polypeptide
  SLT-1 A1 chain tripeptide library construction wherein cysteine 242 has been mutated to an alanine with an example of one N-terminus tag
  MOD_RES (267)..(269): Any amino acid
KGMRSHHHHHHHHIEGRASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHPDXXXAPASRVAR

SEQ ID NO: 31 - protein, 277 aa, Artificial Sequence
  Description of Artificial Sequence: Synthetic polypeptide
  SLT-1 A1 chain tripeptide library construction wherein cysteine 242 has been mutated to an alanine with an example of one N-terminus tag
  MOD_RES (267)..(269): Any amino acid
KGMRSHHHHHHHHRVARASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHPDXXXAPASRVAR

SEQ ID NO: 32 - protein, 300 aa, Artificial Sequence
  Description of Artificial Sequence: Synthetic polypeptide
  7 amino acid random segment inserted between residues 245 and 246 of the SLT-1 A chain less the N-terminus tag
  MOD_RES (246)..(252): Any amino acid
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHXXXXXXXASRVARMASDEFPSMCPADGRVRGITHNKILWDSSTLGAILMRRTISS

SEQ ID NO: 33 - protein, 319 aa, Artificial Sequence
  Description of Artificial Sequence: Synthetic polypeptide
  7 amino acid random segment inserted between residues 245 and 246 of the SLT-1 A chain with an example of one possible N-terminus tag
  MOD_RES (265)..(271): Any amino acid
KGMRSHHHHHHHHIEGRASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHXXXXXXXASRVARMASDEFPSMCPADGRVRGITHNKILWDSSTLGAILMRRTISS

SEQ ID NO: 34 - protein, 319 aa, Artificial Sequence
  Description of Artificial Sequence: Synthetic polypeptide
  7 amino acid random segment inserted between residues 245 and 246 of the SLT-1 A chain with an example of an N-terminus tag
  MOD_RES (265)..(271): Any amino acid
KGMRSHHHHHHHHRVARASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHXXXXXXXASRVARMASDEFPSMCPADGRVRGITHNKILWDSSTLGAILMRRTISS

SEQ ID NO: 35 - protein, 300 aa, Artificial Sequence
  Description of Artificial Sequence: Synthetic polypeptide
  7 amino acid random segment inserted between residues 245 and 246 of the SLT-1 A chain, wherein the cysteine at position 261 has been mutated to an alanine and the cysteine at position 242 has been biotinylated less an N-terminus tag
  MOD_RES (246)..(252): Any amino acid
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHXXXXXXXASRVARMASDEFPSMAPADGRVRGITHNKILWDSSTLGAILMRRTISS

SEQ ID NO: 36 - protein, 319 aa, Artificial Sequence
  Description of Artificial Sequence: Synthetic polypeptide
  7 amino acid random segment inserted between residues 245 and 246 of the SLT-1 A chain, wherein the cysteine at position 261 has been mutated to an alanine and the cysteine at position 242 has been biotinylated with an example
  contd. from above; of one N-terminus tag
  MOD_RES (265)..(271): Any amino acid
KGMRSHHHHHHHHIEGRASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHXXXXXXXASRVARMASDEFPSMAPADGRVRGITHNKILWDSSTLGAILMRRTISS

SEQ ID NO: 37 - protein, 319 aa, Artificial Sequence
  Description of Artificial Sequence: Synthetic polypeptide
  7 amino acid random segment inserted between residues 245 and 246 of the SLT-1 A chain, wherein the cysteine at position 261 has been mutated to an alanine and the cysteine at position 242 has been biotinylated with an example
  contd. from above; of one N-terminus tag
  MOD_RES (265)..(271): Any amino acid
KGMRSHHHHHHHHRVARASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHXXXXXXXASRVARMASDEFPSMAPADGRVRGITHNKILWDSSTLGAILMRRTISS

SEQ ID NO: 38 - protein, 300 aa, Artificial Sequence
  Description of Artificial Sequence: Synthetic polypeptide
  7 amino acid random segment inserted between residues 245 and 246 of the SLT-1 A chain, wherein the cysteine at position 242 has been mutated to an alanine less the N-terminus tag
  MOD_RES (246)..(252): Any amino acid
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHXXXXXXXASRVARMASDEFPSMCPADGRVRGITHNKILWDSSTLGAILMRRTISS

SEQ ID NO: 39 - protein, 319 aa, Artificial Sequence
  Description of Artificial Sequence: Synthetic polypeptide
  7 amino acid random segment inserted between residues 245 and 246 of the SLT-1 A chain, wherein the cysteine at position 242 has been mutated to an alanine with an example of one N-terminus tag
  MOD_RES (265)..(271): Any amino acid
KGMRSHHHHHHHHIEGRASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHXXXXXXXASRVARMASDEFPSMCPADGRVRGITHNKILWDSSTLGAILMRRTISS

SEQ ID NO: 40 - protein, 319 aa, Artificial Sequence
  Description of Artificial Sequence: Synthetic polypeptide
  7 amino acid random segment inserted between residues 245 and 246 of the SLT-1 A chain, wherein the cysteine at position 242 has been mutated to an alanine with an example of one N-terminus tag
  MOD_RES (265)..(271): Any amino acid
KGMRSHHHHHHHHRVARASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHXXXXXXXASRVARMASDEFPSMCPADGRVRGITHNKILWDSSTLGAILMRRTISS

SEQ ID NO: 41 - protein, 300 aa, Artificial Sequence
  Description of Artificial Sequence: Synthetic polypeptide
  7 amino acid random segment inserted between 245 and 246 of the SLT -1 A chain, wherein the cysteine at positions 242 and 261 have been mutated to alanines less the N-terminus tags
  MOD_RES (246)..(252): Any amino acid
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHXXXXXXXASRVARMASDEFPSMAPADGRVRGITHNKILWDSSTLGAILMRRTISS

SEQ ID NO: 42 - protein, 319 aa, Artificial Sequence
  Description of Artificial Sequence: Synthetic polypeptide
  7 amino acid random segment inserted between 245 and 246 of the SLT -1 A chain, wherein the cysteine at positions 242 and 261 have been mutated to alanines with an example of one N-terminus tag
  MOD_RES (265)..(271): Any amino acid
KGMRSHHHHHHHHIEGRASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHXXXXXXXASRVARMASDEFPSMAPADGRVRGITHNKILWDSSTLGAILMRRTISS

SEQ ID NO: 43 - protein, 319 aa, Artificial Sequence
  Description of Artificial Sequence: Synthetic polypeptide
  7 amino acid random segment inserted between 245 and 246 of the SLT -1 A chain, wherein the cysteine at positions 242 and 261 have been mutated to alanines with an example of one N-terminus tag
  MOD_RES (265)..(271): Any amino acid
KGMRSHHHHHHHHRVARASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHXXXXXXXASRVARMASDEFPSMAPADGRVRGITHNKILWDSSTLGAILMRRTISS

SEQ ID NO: 44 - protein, 258 aa, Artificial Sequence
  Description of Artificial Sequence: Synthetic polypeptide
  7 amino acid random segment inserted between residues 245 and 246 of the SLT-1 A1 chain, wherein the cysteine residue at position 242 has been biotinylated less the N-terminus tags
  MOD_RES (246)..(252): Any amino acid
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHXXXXXXXASRVAR

SEQ ID NO: 45 - protein, 277 aa, Artificial Sequence
  Description of Artificial Sequence: Synthetic polypeptide
  7 amino acid random segment inserted between residues 245 and 246 of the SLT-1 A1 chain, wherein the cysteine residue at position 242 has been biotinylated with an example of one N-terminus tag
  MOD_RES (265)..(271): Any amino acid
KGMRSHHHHHHHHIEGRASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHXXXXXXXASRVAR

SEQ ID NO: 46 - protein, 277 aa, Artificial Sequence
  Description of Artificial Sequence: Synthetic polypeptide
  7 amino acid random segment inserted between residues 245 and 246 of the SLT-1 A1 chain, wherein the cysteine residue at position 242 has been biotinylated with an example of one N-terminus tag
  MOD_RES (265)..(271): Any amino acid
KGMRSHHHHHHHHRVARASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHXXXXXXXASRVAR

SEQ ID NO: 47 - protein, 258 aa, Artificial Sequence
  Description of Artificial Sequence: Synthetic polypeptide
  7 amino acid random segment inserted between residues 245 and 246 of the SLT-1 A1 chain, wherein the cysteine residue at position 242 has been mutated to an alanine less the N-terminus tags
  MOD_RES (246)..(252): Any amino acid
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHXXXXXXXASRVAR

SEQ ID NO: 48 - protein, 277 aa, Artificial Sequence
  Description of Artificial Sequence: Synthetic polypeptide
  7 amino acid random segment inserted between residues 245 and 246 of the SLT-1 A1 chain, wherein the cysteine residue at position 242 has been mutated to an alanine with an example of one N-terminus tag
  MOD_RES (265)..(271): Any amino acid
KGMRSHHHHHHHHIEGRASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHXXXXXXXASRVAR

SEQ ID NO: 49 - protein, 277 aa, Artificial Sequence
  Description of Artificial Sequence: Synthetic polypeptide
  7 amino acid random segment inserted between residues 245 and 246 of the SLT-1 A1 chain, wherein the cysteine residue at position 242 has been mutated to an alanine with an example of one N-terminus tag
  MOD_RES (265)..(271): Any amino acid
KGMRSHHHHHHHHRVARASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHXXXXXXXASRVAR

SEQ ID NO: 50 - protein, 293 aa, Artificial Sequence
  Description of Artificial Sequence: Synthetic polypeptide
  Amino acid sequence corresponding to the SLT-1 A region, with cysteine 261 mutated to an alanine
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMAPADGRVRGITHNKILWDSSTLGAILMRRTISS

SEQ ID NO: 51 - protein, 312 aa, Artificial Sequence
  Description of Artificial Sequence: Synthetic polypeptide
  Amino acid sequence corresponding to the SLT-1 A region, with cysteine 261 mutated to an alanine, and with an example of one N-terminus tag
KGMRSHHHHHHHHIEGRASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMAPADGRVRGITHNKILWDSSTLGAILMRRTISS

SEQ ID NO: 52 - protein, 312 aa, Artificial Sequence
  Description of Artificial Sequence: Synthetic polypeptide
  Amino acid sequence corresponding to the SLT-1 A region, with cysteine 261 mutated to an alanine, and with an example of one N-terminus tag
KGMRSHHHHHHHHRVARASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMAPADGRVRGITHNKILWDSSTLGAILMRRTISS

SEQ ID NO: 53 - protein, 293 aa, Artificial Sequence
  Description of Artificial Sequence: Synthetic polypeptide
  Amino acid sequence corresponding to SLT-1 A region, with cysteine 242 mutated to alanine
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHASRVARMASDEFPSMCPADGRVRGITHNKILWDSSTLGAILMRRTISS

SEQ ID NO: 54 - protein, 312 aa, Artificial Sequence
  Description of Artificial Sequence: Synthetic polypeptide
  Amino acid sequence corresponding to SLT-1 A region, with cysteine 242 mutated to alanine with an example of one N-terminus tag
KGMRSHHHHHHHHIEGRASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHASRVARMASDEFPSMCPADGRVRGITHNKILWDSSTLGAILMRRTISS

SEQ ID NO: 55 - protein, 312 aa, Artificial Sequence
  Description of Artificial Sequence: Synthetic polypeptide
  Amino acid sequence corresponding to SLT-1 A region, with cysteine 242 mutated to alanine with an example of one N-terminus tag
KGMRSHHHHHHHHRVARASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHASRVARMASDEFPSMCPADGRVRGITHNKILWDSSTLGAILMRRTISS

SEQ ID NO: 56 - protein, 293 aa, Artificial Sequence; identical to SEQ ID NO: 53
  Description of Artificial Sequence: Synthetic polypeptide
  Amino acid sequence corresponding to the SLT-1 A region, with cysteines 242 and 261 mutated to alanines

SEQ ID NO: 57 - protein, 312 aa, Artificial Sequence; identical to SEQ ID NO: 54
  Description of Artificial Sequence: Synthetic polypeptide
  Amino acid sequence corresponding to the SLT-1 A region, with cysteines 242 and 261 mutated to alanines with an example of one possible N-terminus tag

SEQ ID NO: 58 - protein, 312 aa, Artificial Sequence; identical to SEQ ID NO: 55
  Description of Artificial Sequence: Synthetic polypeptide
  Amino acid sequence corresponding to the SLT-1 A region, with cysteines 242 and 261 mutated to alanines with an example of one N-terminus tag

SEQ ID NO: 59 - protein, 251 aa, Artificial Sequence
  Description of Artificial Sequence: Synthetic polypeptide
  Amino acid sequence corresponding to the SLT-1 A1 region
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAR

SEQ ID NO: 60 - protein, 270 aa, Artificial Sequence
  Description of Artificial Sequence: Synthetic polypeptide
  Amino acid sequence corresponding to the SLT-1 A1 region with an example of one possible N-terminus tag
KGMRSHHHHHHHHIEGRASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAR

SEQ ID NO: 61 - protein, 270 aa, Artificial Sequence
  Description of Artificial Sequence: Synthetic polypeptide
  Amino acid sequence corresponding to the SLT-1 A1 region with an example of one N-terminus tag
KGMRSHHHHHHHHRVARASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAR

SEQ ID NO: 62 - protein, 251 aa, Artificial Sequence
  Description of Artificial Sequence: Synthetic polypeptide
  Amino acid sequence corresponding with the SLT-1 A1 region, with cysteine 242 mutated to an alanine
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHASRVAR

SEQ ID NO: 63 - protein, 270 aa, Artificial Sequence
  Description of Artificial Sequence: Synthetic polypeptide
  Amino acid sequence corresponding with the SLT-1 A1 region, with cysteine 242 mutated to an alanine with an example of one N-terminus tag
KGMRSHHHHHHHHIEGRASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHASRVAR

SEQ ID NO: 64 - protein, 270 aa, Artificial Sequence
  Description of Artificial Sequence: Synthetic polypeptide
  Amino acid sequence corresponding with the SLT-1 A1 region, with cysteine 242 mutated to an alanine with an example of one N-terminus tag
KGMRSHHHHHHHHRVARASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHASRVAR

SEQ ID NO: 65 - DNA, 21 bp, Artificial Sequence
  Description of Artificial Sequence: Synthetic oligonucleotide
  DNA sequences of randomly picked clones from the SLT 1 A chain tripeptide library. MUC1 epitope
ccagacacgcgaccagctcca

SEQ ID NO: 66 - DNA, 21 bp, Artificial Sequence
  Description of Artificial Sequence: Synthetic oligonucleotide
  DNA sequences of randomly picked clones from the SLT 1 A chain tripeptide library. Mutant #1
ccagacgggatcggggctcca

SEQ ID NO: 67 - DNA, 21 bp, Artificial Sequence
  Description of Artificial Sequence: Synthetic oligonucleotide
  DNA sequences of randomly picked clones from the SLT 1 A chain tripeptide library. Mutant #2
ccagacctggagatggctcca

SEQ ID NO: 68 - DNA, 21 bp, Artificial Sequence
  Description of Artificial Sequence: Synthetic oligonucleotide
  sequences of randomly picked clones from the SLT 1 A chain tripeptide library. Mutant #3
ccagacccccgtggggctcca

SEQ ID NO: 69 - DNA, 21 bp, Artificial Sequence
  Description of Artificial Sequence: Synthetic oligonucleotide
  DNA sequences of randomly picked clones from the SLT 1 A chain tripeptide library. Mutant #4
ccagacgacgacttggctcca

SEQ ID NO: 70 - DNA, 21 bp, Artificial Sequence
  Description of Artificial Sequence: Synthetic oligonucleotide
  DNA sequences of randomly picked clones from the SLT 1 A chain tripeptide library. Mutant #5
ccagacgtccggtgggctcca

SEQ ID NO: 71 - DNA, 21 bp, Artificial Sequence
  Description of Artificial Sequence: Synthetic oligonucleotide
  DNA sequences of randomly picked clones from the SLT 1 A chain tripeptide library. Mutant #6
ccagaccagcgctgggctcca

SEQ ID NO: 72 - DNA, 21 bp, Artificial Sequence
  Description of Artificial Sequence: Synthetic oligonucleotide
  DNA sequences of randomly picked clones from the SLT 1 A chain tripeptide library. Mutant #7
ccagacctcaggatggctcca

SEQ ID NO: 73 - DNA, 21 bp, Artificial Sequence
  Description of Artificial Sequence: Synthetic oligonucleotide
  DNA sequences of randomly picked clones from the SLT 1 A chain tripeptide library. Mutant #8
ccagactcccaggaggctcca

SEQ ID NO: 74 - DNA, 21 bp, Artificial Sequence
  Description of Artificial Sequence: Synthetic oligonucleotide
  DNA sequences of randomly picked clones from the SLT 1 A chain tripeptide library. Mutant #9
ccagactccgaccccgctcca

SEQ ID NO: 75 - DNA, 21 bp, Artificial Sequence
  Description of Artificial Sequence: Synthetic oligonucleotide
  DNA sequences of randomly picked clones from the SLT 1 A chain tripeptide library. Mutant #41
ccagacacgcgccccgctcca

SEQ ID NO: 76 - protein, 27 aa, Artificial Sequence
  Description of Artificial Sequence: Synthetic peptide
  Amino acid sequence alignment of randomly selected clones from the SLT 1 A chain tripeptide library. MUC1 epitope
CHHHPDTRPAPASRVARMASDEFPSMC

SEQ ID NO: 77 - protein, 27 aa, Artificial Sequence
  Description of Artificial Sequence: Synthetic peptide
  Amino acid sequence alignment of randomly selected clones from the SLT 1 A chain tripeptide library. Mutant #1
CHHHPDGIGAPASRVARMASDEFPSMC

SEQ ID NO: 78 - protein, 27 aa, Artificial Sequence
  Description of Artificial Sequence: Synthetic peptide
  Amino acid sequence alignment of randomly selected clones from the SLT 1 A chain tripeptide library. Mutant #2
CHHHPDLQMAPASRVARMASDEFPSMC

SEQ ID NO: 79 - protein, 27 aa, Artificial Sequence
  Description of Artificial Sequence: Synthetic peptide
  Amino acid sequence alignment of randomly selected clones from the SLT 1 A chain tripeptide library. Mutant #3
CHHHPDPRGAPASRVARMASDEFPSMC

SEQ ID NO: 80 - protein, 27 aa, Artificial Sequence
  Description of Artificial Sequence: Synthetic peptide
  Amino acid sequence alignment of randomly selected clones from the SLT 1 A chain tripeptide library. Mutant #4
CHHHPDDDLAPASRVARMASDEFPSMC

SEQ ID NO: 81 - protein, 27 aa, Artificial Sequence
  Description of Artificial Sequence: Synthetic peptide
  Amino acid sequence alignment of randomly selected clones from the SLT 1 A chain tripeptide library. Mutant #5
CHHHPDVRWAPASRVARMASDEFPSMC

SEQ ID NO: 82 - protein, 27 aa, Artificial Sequence
  Description of Artificial Sequence: Synthetic peptide
  Amino acid sequence alignment of randomly selected clones from the SLT 1 A chain tripeptide library. Mutant #6
CHHHPDQRLAPASRVARMASDEFPSMC

SEQ ID NO: 83 - protein, 27 aa, Artificial Sequence
  Description of Artificial Sequence: Synthetic peptide
  Amino acid sequence alignment of randomly selected clones from the SLT 1 A chain tripeptide library. Mutant #7
CHHHPDLRMAPASRVARMASDEFPSMC

SEQ ID NO: 84 - protein, 27 aa, Artificial Sequence
  Description of Artificial Sequence: Synthetic peptide
  Amino acid sequence alignment of randomly selected clones from the SLT 1 A chain tripeptide library. Mutant #8
CHHHPDSQEAPASRVARMASDEFPSMC

SEQ ID NO: 85 - protein, 27 aa, Artificial Sequence
  Description of Artificial Sequence: Synthetic peptide
  Amino acid sequence alignment of randomly selected clones from the SLT 1 A chain tripeptide library. Mutant #9
CHHHPDSDPAPASRVARMASDEFPSMC

SEQ ID NO: 86 - protein, 27 aa, Artificial Sequence
  Description of Artificial Sequence: Synthetic peptide
  Amino acid sequence alignment of randomly selected clones from the SLT 1 A chain tripeptide library. Mutant #41
CHHHPDTRPAPASRVARMASDEFPSMC

SEQ ID NO: 87 - protein, 7 aa, Artificial Sequence
  Description of Artificial Sequence: Synthetic peptide
  MUC-1 sequence
PDTRPAP

SEQ ID NO: 88 - protein, 7 aa, Artificial Sequence
  Description of Artificial Sequence: Synthetic peptide
  MUC-1 sequence
  MOD_RES (3)..(5): Any amino acid
PDXXXAP

SEQ ID NO: 89 - protein, 6 aa, Artificial Sequence
  Description of Artificial Sequence: Synthetic 6xHis tag
HHHHHH

SEQ ID NO: 90 - protein, 8 aa, Artificial Sequence
  Description of Artificial Sequence: Synthetic 8xHis tag
HHHHHHHH

SEQ ID NO: 91 - protein, 19 aa, Artificial Sequence
  Description of Artificial Sequence: Synthetic peptide
KGMRSHHHHHHHHRVARAS

SEQ ID NO: 92 - protein, 19 aa, Artificial Sequence
  Description of Artificial Sequence: Synthetic peptide
KGMRSHHHHHHHHIEGRAS

SEQ ID NO: 93 - protein, 319 aa, Artificial Sequence
  Description of Artificial Sequence: Synthetic polypeptide
  MOD_RES (267)..(269): Any amino acid
KGMRSHHHHHHHHIEGRASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHPDXXXAPASRVARMASDEFPSMAPADGRVRGITHNKILWDSSTLGAILMRRTISS

SEQ ID NO: 94 - protein, 258 aa, Artificial Sequence
  Description of Artificial Sequence: Synthetic polypeptide
  MOD_RES (248)..(250): Any amino acid
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHPDXXXAPASRVAR

SEQ ID NO: 95 - protein, 277 aa, Artificial Sequence
  Description of Artificial Sequence: Synthetic polypeptide
  MOD_RES (267)..(269): Any amino acid
KGMRSHHHHHHHHIEGRASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHPDXXXAPASRVAR

SEQ ID NO: 96 - protein, 258 aa, Artificial Sequence
  Description of Artificial Sequence: Synthetic polypeptide
  MOD_RES (248)..(250): Any amino acid
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNAHHHPDXXXAPASRVAR